(19) United States
(12) Patent Application Publication
(10) Pub. No.: US 2019/0325316 A1
(43) Pub. Date: Oct. 24, 2019

(54) APPARATUS AND METHODS FOR PROGRAM SYNTHESIS USING GENETIC ALGORITHMS

(71) Applicant: Intel Corporation, Santa Clara, CA (US)

(72) Inventors: Todd A. Anderson, Hillsboro, OR (US); Mohammad Mejbah Ul Alam, Milpitas, CA (US); Shengtian Zhou, Palo Alto, CA (US); Justin Gottschlich, Santa Clara, CA (US)

(21) Appl. No.: 16/457,133

(22) Filed: Jun. 28, 2019

Related U.S. Application Data
(60) Provisional application No. 62/849,120, filed on May 16, 2019.

(51) Int. Cl. G06N 3/08; G16H 20/30

(52) U.S. Cl. CPC G06N 3/086; G16H 20/30

(57) ABSTRACT

Example apparatus and methods for program synthesis using genetic algorithms are disclosed herein. An example apparatus includes a program length predictor to predict a length of a first program by executing a first neural network model, a program generator to generate a candidate program having a length corresponding to the predicted length, a candidate program analyzer to generate a fitness score for the candidate program by executing a second neural network model and to identify the first candidate program for use in a breeding operation relative a second candidate program based on the fitness score, and a genetic program generator to perform the breeding operation with at least one of the first candidate program or the second candidate program to generate an evolved candidate program.

### RELATED APPLICATION

This patent claims the benefit of, and priority to, U.S. Provisional Patent Application Ser. No. 62/849,120, entitled “Apparatus and Methods for Program Synthesis Using Genetic Algorithms” and filed on May 16, 2019. U.S. Provisional Patent Application Ser. No. 62/849,120 is hereby incorporated by reference in its entirety.

### FIELD OF THE DISCLOSURE

This disclosure relates generally to program synthesis and, more particularly, to apparatus and methods for program synthesis using genetic algorithms.

### BACKGROUND

Program synthesis involves the automatic generation (e.g., by a computer) of a program (e.g., source code) to satisfy particular criteria, such as an input and a corresponding known output. Program synthesis can be based on genetic algorithms, in which random programs are generated and certain ones of the programs are iteratively combined to create a new program in an effort to find a composite program that satisfies the target input and output.

### BRIEF DESCRIPTION OF THE DRAWINGS

FIG. 1 is a flow diagram illustrating execution of a known genetic algorithm for program synthesis in accordance with teachings of this disclosure.

FIG. 1A is a block diagram of an example system including an artificial genetic algorithm programmer to perform genetic programming based on a genetic algorithm and including one or more computing systems for training neural networks to generate models for use during genetic programming in accordance with teachings of this disclosure.

FIG. 2 is a block diagram of an example implementation of the candidate program analyzer of the example system of FIG. 1A.

FIG. 3 is a flowchart representative of example machine readable instructions that, when executed by a first computing system of the example system of FIG. 1A, cause the first computing system to train a neural network to predict program length.

FIG. 4 is flowchart representative of example machine readable instructions that, when executed by a second computing system of the example system of FIG. 1A, cause the second computing system to train a neural network to generate a fitness function model.

FIG. 5 is flowchart representative of example machine readable instructions that may be executed to implement block 402 of FIG. 4.

FIG. 5A is a flowchart representative of example machine readable instructions that may be executed to implement block 502 of FIG. 5.

FIG. 6 is flowchart representative of example machine readable instructions that, when executed by a third computing system of the example system of FIG. 1A, cause the third computing system to train a neural network to select a fitness function model.

FIG. 7 is a flowchart of example machine readable instructions that, when executed by the example system of FIG. 1A, cause the example genetic algorithm programmer of FIG. 1A to generate program(s) based on genetic programming.

FIG. 7A is a flowchart representative of example machine readable instructions that may be executed to implement block 700 of FIG. 7.

FIG. 8 is a flowchart representative of example machine readable instructions that, when executed by any of the first computing system, the second computing system, and/or the third computing system of the example system of FIG. 1A, cause the first computing system, the second computing system, and/or the third computing system to filter randomly generated programs from training data.

FIG. 9 is a block diagram of an example processing platform structured to execute the instructions of FIGS. 3 and/or 8 to implement the example first computing system of FIG. 1A.

FIG. 10 is a block diagram of an example processing platform structured to execute the instructions of FIGS. 4, 5, 5A, and/or 8 to implement the example second computing system of FIG. 1A.

FIG. 11 is a block diagram of an example processing platform structured to execute the instructions of FIGS. 6 and/or 8 to implement the example third computing system of FIG. 1A.

FIG. 12 is a block diagram of an example processing platform structured to execute the instructions of FIGS. 7 and/or 7A to implement the example genetic algorithm programmer of FIGS. 1 and 2.

The figures are not to scale. In general, the same reference numbers will be used throughout the drawing(s) and accompanying written description to refer to the same or like parts.

Descriptors “first,” “second,” “third,” etc. are used herein when identifying multiple elements or components which may be referred to separately. Unless otherwise specified or understood based on their context of use, such descriptors are not intended to impute any meaning of priority, physical order or arrangement in a list, or ordering in time but are merely used as labels for referring to multiple elements or components separately for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for ease of referencing multiple elements or components.

### DETAILED DESCRIPTION

Known program synthesis involves the automatic generation of a program (e.g., source code) by a computer via artificial intelligence. The computer automatically generates the program to satisfy particular criteria, such as an input and a corresponding known output. Program synthesis can be based on genetic algorithm programming, in which random programs are generated as candidate programs and certain ones of the candidate programs are iteratively bred (e.g., combined, mutated) to create new programs in an effort to find program(s) that satisfy the target input and output.

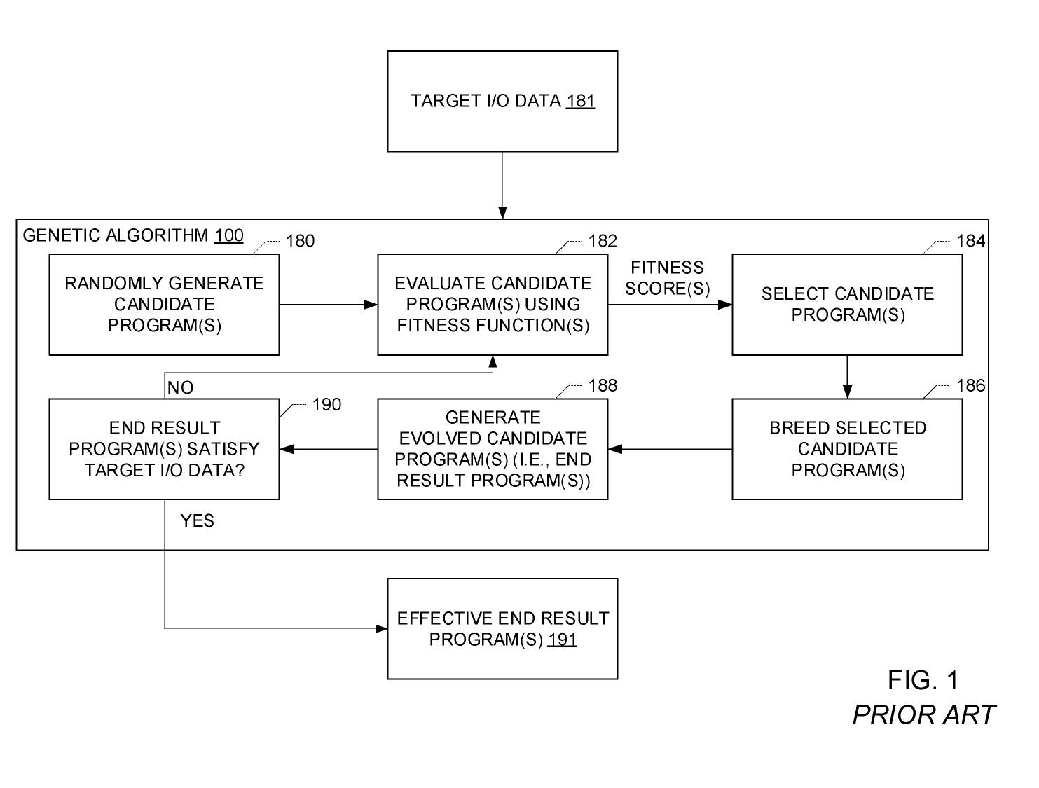

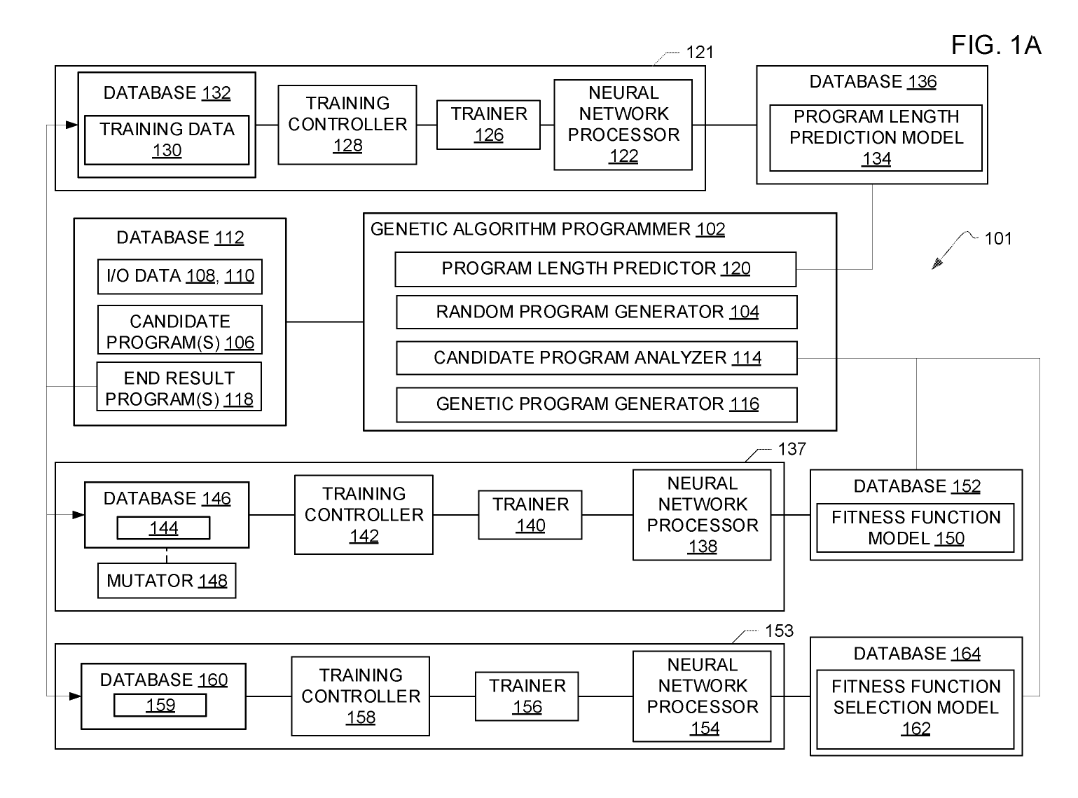

Genetic algorithms are based on the evolutionary idea of natural selection in which the fittest programs are selected for reproduction and genetically bred to produce offspring program(s). FIG. 1 is a flow diagram illustrating execution of a known genetic algorithm 100 for program synthesis. The known genetic algorithm 100 of FIG. 1 can be implemented by one or more processors (e.g., semiconductor-based device implementing artificial genetic algorithm programmer(s)). In FIG. 1, candidate programs are randomly generated (block 180). (As used herein, random includes pseudo random and/or truly random unless otherwise specified). The randomly generated candidate program(s) are evaluated using fitness function(s) to determine a fitness score for each candidate program (block 182). The fitness score represents, for example, the ability of a candidate program to satisfy particular (e.g., target) input/output criteria 181. For instance, for a given known input and corresponding known output, a candidate program is run based on the known input to determine if the candidate program produces the corresponding known output. A fitness score is assigned to the candidate program based on the behavior of the candidate program with respect to the known input/output data. For instance, the fitness score may be based on whether the candidate program produces the known output. If the program does not produce the known output, the fitness score may be further adjusted based on how close the output produced by the program is to the known output.

In the known genetic algorithm 100 of FIG. 1, certain ones of the randomly generated candidate programs are selected based on the fitness scores for use in breeding operation(s) to generate new program(s) (block 184). Candidate program(s) associated with fitness score(s) indicating that the program(s) perform better than other candidate program(s) in satisfying the target input/output data are more likely to be selected for breeding, as such programs are more likely to produce end result program(s) that satisfy the target input/output criteria. Breeding operations can include mutation in which one or more parameters (e.g., one or more instructions, one or more values, one or more variables, one or more data structures, one or more objects, one or more loops, etc.) of a selected candidate program are modified. Another example breeding operation includes crossover, in which parameter(s) e.g., one or more instructions, one or more values, one or more variables, one or more data structures, one or more objects, one or more loops, etc.) of different candidate programs are combined (e.g., swapped, merged, added, removed, etc.) to generate new program(s).

As a result of the breeding operation(s), evolved candidate program(s) (also referred to herein as end result program(s)) are generated (blocks 186, 188). In FIG. 1, the end results program(s) are evaluated to determine whether the end result program(s) satisfy the target input/output criteria (block 190). As used herein, an end result program is considered to satisfy the target input/output criteria and, thus, is considered an effective end result program 191, if for all given inputs, the end result program provides corresponding desired outputs. If none of the end result program(s) satisfies the target input/output data, then additional iterations of the known genetic algorithm 100 are performed in an effort to identify effective end result program(s) (blocks 182-188). Breeding selected candidate programs and evaluating the resulting offspring program(s) can be performed over multiple generations to improve the effectiveness of the offspring program(s) in meeting the target input/output criteria.

Thus, known program synthesis based on genetic programming includes generating random programs that are iteratively selected for use in generating new programs. There can be more than one program that provides a solution to given input/output example. Thus, as a result of program synthesis, one or more effective end result programs can be generated that satisfy the target input/output criteria. As discussed above, as used herein, an end program can be considered to be an effective end program if for all given inputs, the end result program provides corresponding desired outputs. However, other structural or timing based characteristics of an effective end result program, such as a length of the end result program, may be unknown.

A length of a program can be based on a number of statements (e.g., instructions) in the program. If a desired length of a candidate program to be used in genetic programming differs substantially from the length of an effective end result program developed using genetic programming, then the artificial genetic programmer executing, for instance, the known genetic algorithm 100 of FIG. 1, may not be able to identify the end result program as an effective end result program because the candidate program is not satisfactory for the intended context. In other examples, many iterations of the known genetic algorithm 100 are run until an effective end result program that satisfies all requirements of the intended application context is ultimately identified (e.g., if the initial length of the randomly generated program(s) differ substantially from the length of the effective end result program, the known genetic algorithm 100 may be repeated to find a better solution). Thus, some known genetic programming techniques can result in inefficient use of processing resources and time. In some known instances, a user may provide a random program generator with a particular program length to tune the random program generator to create candidate programs having a certain length. However, such prior knowledge with respect to program length is typically not available when producing programs using program synthesis.

As mentioned above in connection with the known genetic algorithm 100 of FIG. 1, programs can be generated based on genetic algorithms by breeding randomly generated candidate programs (i.e., parent programs) to create new program(s) (i.e., offspring program(s)). Example breeding operations include crossover and/or mutation of instructions, objects, variables, data, and/or data structures of the parent programs to generate the offspring program(s). To avoid breeding candidate programs that are not likely to facilitate production of effective end result program(s), a fitness function may be used to measure how close, for instance, a candidate program is to providing the correct known output for a given input. As discussed above in connection with the known genetic algorithm 100 of FIG. 1, the fitness function is used to calculate a fitness score that indicates how close the candidate program is to correctly providing the desired output. Based on the fitness or correctness score, the candidate program may or may not be selected for breeding with other candidate program(s). Thus, the particular fitness function that is used to assess the candidate program can affect whether or not a program is used in subsequent genetic programming iterations.

In some known genetic algorithms, a single fitness function is used to evaluate all candidate programs, irrespective of the target input/output data 181. However, one fitness function may not be effective (e.g., optimal) in all such cases in view of the different possible target input/output data 181. Indeed, use of a single fitness function to assess the candidate programs can negatively affect the selection of candidate programs that are used in breeding operation(s) (e.g., mutation, crossover). For instance, use of a single fitness function can result in the selection of certain candidate programs for breeding. However, those programs do not perform as well in producing the desired output in ways not tested by the single fitness function despite satisfying the fitness function. Conversely, other candidate programs that may not satisfy the factors tested by the single fitness function may be discarded even though those programs would perform better in producing the desired output than one or more of the candidate programs that did pass the test of the fitness function. Also, some known fitness functions are based on linear distance metrics that measure differences between a known program's output and a candidate program's output to evaluate the candidate program, such as the Manhattan distance or edit distance. However, distance metrics such as the Manhattan distance may not accurately evaluate the candidate program(s) and/or may not accurately detect patterns in the candidate program(s) and/or the outputs generated. For instance, a candidate program that is only one change away from providing a desired output may erroneously be given a low measure of similarity with respect to the known output by a distance-based fitness function such as the Manhattan distance. Fitness functions written by human users also tend to be based on superficial similarities between outputs and, thus, oversimplify the fitness evaluation.

Disclosed herein are example apparatus and methods to provide for efficient genetic programming. Some examples disclosed herein include a program length predictor to predict a length of an effective end result program. The predicted length is used to generate candidate programs that are used to produce end result program(s), wherein such selected end result program(s) correctly provide an output for given input(s) via genetic programming. In examples disclosed herein, a neural network is trained using sample programs having known lengths and associated program inputs and outputs to predict the lengths of new programs that are able to produce such known outputs based on known inputs. A program length prediction model is generated based on the training. In examples disclosed herein, the program length predictor executes the program length prediction model to determine a predicted length of an end result program for a given set of target input(s) and output(s). The predicted program length is used by a random program generator to produce candidate program(s) that have a length corresponding to the predicted program length (e.g., within +/−1 instructions, within +/−2 instructions) or near the predicted program length (e.g., based on a predicted program length distribution). Alternatively, the random program generator discards programs that do not satisfy the length requirements without labeling them as candidate programs and/or with adding them to the set of candidate programs. As a result, the candidate program(s) generated during breeding based on the genetic algorithm(s) have lengths that are similar to the predicted program length (e.g., within +/−1 instructions). Thus, the efficiency of the artificial genetic algorithm programmer in identifying effective end result program(s) is increased as the candidate program size is bounded or limited based on the predicted end result program length.

Also disclosed herein are example apparatus and methods for generating fitness functions based on machine learning. In examples disclosed herein, a neural network is used to generate a fitness function model that can be used to evaluate the candidate programs. In some examples, known sample programs that generate correct results for particular input/output examples are mutated (e.g., modified) to introduce errors into the programs such that the programs may no longer produce the desired outputs for the given inputs. The mutated programs are run to generate outputs and to create training data. The resulting training data includes the correct outputs generated by the programs without any errors in the program, the outputs generated by the mutated programs, and fitness scores that measure how close the mutated programs are to generating the correct output. In examples disclosed herein, a neural network is trained based on the training data to produce a fitness function model. During the inference phase, the neural network-derived fitness function model is used to estimate the number of mistakes in a candidate program. In some examples, the fitness function model is run for multiple input/output pairs and the fitness score of the candidate program for each input/output pair are combined (e.g., averaged) to create a fitness or correctness estimate for the candidate program. The correctness estimate for the candidate program is used during genetic programming to determine if the candidate programs should be combined (e.g., partly combined) with other candidate programs (e.g., via crossover) and/or mutated. This neural network-based fitness function more accurately determines a fitness score for a candidate program as compared to, for instance, distance metrics (e.g., Manhattan distance) and/or human-written fitness functions. For example, the example neural network-based fitness function 150 may identify patterns (e.g., non-linear patterns) between the inputs and the outputs that may not be captured by known fitness functions (e.g., known fitness functions that are not generated using machine learning).

Also disclosed herein are example apparatus and methods for selecting a fitness function from a collection of possible fitness functions to use for a given candidate program. Although as mentioned above, neural network-derived fitness functions can provide for more accurate analysis of the candidate programs, in some examples, it may be desirable to additionally or alternatively use another type of fitness function, such a distance metric. In examples disclosed herein, a neural network is trained to select a fitness function to evaluate a program from among a plurality of possible known fitness functions and based on criteria such as specifications of a desired program or end use applications. For example, the neural network can be trained based on various input/output examples to predict an optimal fitness function for a particular candidate program. In some examples, the training data can be based on particular types of data, such as financial data, scientific data, etc. Thus, in examples disclosed herein, the neural networks can be tuned to generate models that are developed in view of target data of interest.

FIG. 1A is a block diagram of an example system 101 for generating programs based on genetic programming (e.g., by implementing a genetic algorithm). The example system 101 of FIG. 1A includes an artificial genetic algorithm programmer 102. The example artificial genetic algorithm programmer 102 of FIG. 1A is implemented by a logic circuit such as, for example, a hardware processor (e.g., a semiconductor based (e.g., silicon based) integrated circuit). However, any other type of circuitry may additionally or alternatively be used such as, for example, one or more analog or digital circuit(s), logic circuits, programmable processor(s), application specific integrated circuit(s) (ASIC(s)), programmable logic device(s) (PLD(s)), field programmable logic device(s) (FPLD(s)), digital signal processor(s) (DSP(s)), etc. The example genetic algorithm programmer 102 of FIG. 1A applies one or more genetic algorithms to iteratively generate programs in view of target input(s) and corresponding output(s) and to evaluate the generated programs to produce end result program(s) that satisfy the target input and output data.

The example genetic algorithm programmer 102 of FIG. 1A includes a random program generator 104. In this example, the random program generator 104 implements means for generating candidate program(s) 106. As disclosed herein, the random program generator 104 of FIG. 1 A generates the candidate program(s) 106 having particular length(s) (e.g., a number of statements (i.e., machine readable instructions)) based on a predicted length of an effective end result program, as determined using a neural network.

The random program generator 104 generates the random program(s) 106 using one or more processing languages. In some examples, the random program generator 104 of FIG. 1A generates the random program(s) 106 based on a list-processing oriented language. The data types of the example language can include integers and/or lists of integers. The example language can include a certain number of operations defined in the language. For instance, the programming language can include 40 operations defined in the language, where the operations are numbered from 0 to 40. The operations can be associated with parameters. For example, some of the operations operate on two lists (e.g., element-wise addition) to produce a new list whereas other operations operate based on one list and one integer (e.g., selection of a given index from a list). The example operations can include other types of parameters. In some such examples, statements of the program being generated reference the output of a previous statement by relative index and type (e.g., for a given function parameter, a particular statement uses the second most recent list output). The example language is defined such that when each statement in the language is based on one of the (e.g., 40) defined operations, the program will be valid by construction and can run without error.

For example, to add a new statement to a program based on the example list-processing oriented language, the random program generator 104 generates a random number between 0 and the number of operations (e.g., 40). The random program generator 104 identifies the operation associated with the random number and the parameter(s) associated with the operation. The random program generator 104 adds the operation and the parameter(s) to the candidate program as a statement of the program. The example random program generator 104 continues to add operations and corresponding operation parameters to the program until the desired program length is reached.

In the example of FIG. 1A, the candidate program(s) 106 generated by the random program generator 104 are stored in a database 112. The example database 112 also stories known input data 108 and corresponding known output data 110. The input data 108 and the output data 110 can be defined based on user inputs (e.g., which can define end use applications of the program(s)). The input and output data 108, 110 define parameters. The genetic algorithm programmer 102 attempts to identify a program that satisfies those parameters. The example database 112 of the illustrated example of FIG. 1A is implemented by any memory, storage device and/or storage disc for storing data such as, for example, flash memory, magnetic media, optical media, etc. Furthermore, the data stored in the example database 112 may be in any data format such as, for example, binary data, comma delimited data, tab delimited data, structured query language (SQL) structures, image data, etc. While in the illustrated example database 112 is illustrated as a single element, the database 112 and/or any other data storage elements described herein may be implemented by any number and/or type(s) of memories. In some examples, the genetic algorithm programmer 102 includes the database 112. In other examples, the database 112 is located external to the genetic algorithm programmer 102 in a location accessible to the genetic algorithm programmer 102 as shown in FIG. 1A.

The example genetic algorithm programmer 102 of FIG. 1A includes a candidate program analyzer 114. In this example, the candidate program analyzer 114 implements means for evaluating the candidate program(s) 106. The candidate program analyzer 114 evaluates the candidate program(s) 106 generated by the random program generator 104 to assess a fitness (e.g., correctness) of the candidate program(s) 106 in producing output(s) relative to the target output(s) 110. As disclosed below, the candidate program analyzer 114 also assess the fitness of evolved candidate programs generated as a result of breeding operations with respect to the target input/output data 108, 110 to determine effective end programs. The example candidate program analyzer 114 of FIG. 1A assesses the correctness of the respective candidate programs 106 using a fitness function. As disclosed herein, in the example of FIG. 1A, the candidate program analyzer 114 executes the fitness function generated based on a neural network model to evaluate a given candidate program 106. Based on the evaluation of the candidate program(s) 106 using the neural network-derived fitness function, the candidate program analyzer 114 identifies the candidate program(s) 106 that are to be assigned a higher probability of being used to generate end result program(s) based on genetic programming (e.g., by breeding of the candidate program(s) 106 using operations such as crossover and/or mutation). As a result of identifying the high performing candidate program(s) 106 based on the fitness scores, the candidate program analyzer 114 creates a biased distribution of the candidate program(s) 106 that should be selected for genetic breeding.

The example genetic algorithm programmer 102 of FIG. 1A includes a genetic program generator 116. In this example, the genetic program generator 116 implements means for performing breeding operation(s) to generate new program(s) or end result program(s) based on the candidate program(s) 106. The example genetic program generator 116 of FIG. 1A generates the new program(s) or end result program(s) based on the candidate program(s) 106 and via one or more breeding operations. The breeding operation(s) implemented by the genetic program generator 116 of FIG. 1 A can include, for example, mutation (e.g., alteration) of the candidate program(s) 106 and/or crossover (e.g., combination) of the candidate program(s) 106. At least some of the evolved candidate programs are added to and/or replace the candidate programs in a prior set of candidate programs. In this way, the set of candidate programs is iteratively adapted. For example, the genetic program generator 116 generates a certain number of evolved candidate programs (e.g., a predefined number) per generation, such as 100 new candidate programs. For each new evolved candidate program, the genetic program generator 116 randomly decides if the evolved candidate program will be generated through crossover or mutation. If the genetic program generator 116 randomly decides to generate the evolved candidate program via crossover (e.g., swapping of parameter(s) of two programs), the genetic program generator 116 selects two candidate program(s) 106 at random from the previous generation of candidate programs. In the example of FIG. 1A, the distribution of randomness with respect to the genetic program generator 116 selecting two candidate programs is biased toward the candidate program(s) 106 with high performing fitness scores as identified by the example candidate program analyzer 114. If the genetic program generator 116 randomly decides to generate the evolved candidate program via mutation (e.g. modification of program parameter(s)), the genetic program generator 116 randomly selects one candidate program 106 to undergo mutation. The random selection of the candidate program 106 for mutation is biased toward the candidate program(s) 106 with high fitness scores as evaluated by the example candidate program analyzer 114. As a result, poorly performing candidate program(s) 106 (e.g., as determined by the candidate program analyzer 114 are less likely to be used in creating the evolved candidate program(s) and in some instances, are ultimately replaced via the iterative adaption of the candidate program set. The evolved candidate program(s) are stored in the database 112.

In the example of FIG. 1A, the candidate program analyzer 114 evaluates the new evolved program(s) generated by the genetic program generator 116. For example, the candidate program analyzer 114 determines if a candidate program generated by the genetic program generator 116 should be selected as an end result program because it satisfies the target input/output data 108, 110 such that for a particular input 108, the end result program produces the correct output 110. For example, each evolved candidate program is executed to process the input data 108 to produce a corresponding output. For a given input, the output produced by a respective evolved candidate program is compared to the corresponding target output data 110. If the candidate program analyzer 114 determines that a particular program generated by the genetic program generator 116 satisfies all of the example outputs 110 for the corresponding inputs 108, the candidate program analyzer 114 determines that the program is an effective end result program 118. The genetic algorithm programmer 102 identifies each effective end result program 118 as a solution to the target input/output data 108, 110. If none of the end result program(s) generated by the genetic program generator 116 satisfy the input/output examples 108, 110, then the genetic algorithm programmer 102 performs additional iteration(s) of breeding and evaluating candidate programs (e.g., evolved candidate programs) to identify solution(s) to the target input/output criteria 108, 110 (i.e., one or more programs that satisfactorily generate correct outputs for the inputs). In some examples, the genetic algorithm programmer 102 performs multiple iterations in an effort to generate effective end result program(s) until a threshold number of iterations (e.g., a predefined number) is reached. In some such examples, the genetic algorithm programmer 102 may or may not identify solution(s) to the target input/output data 108, 110 within the threshold number of iterations.

In the example of FIG. 1A, machine learning is used to improve efficiency of the genetic algorithm programmer 102 identifying the effective end result program(s) 118. As disclosed herein, in some examples, the random program generator 104 generates the candidate program(s) 106 having a respective program length based on a length (or mean length or distribution) that is predicted by a program length predictor 120 of the genetic algorithm programmer 102. In this example, the program length predictor 120 implements means for predicting a length of an effective end result program. The program length predictor 120 predicts the length based on a neural network model, where the length is predicted for an effective end result program that satisfies the target input/output criteria. In some examples, the candidate program analyzer 114 uses a fitness function generated via neural network training to assess the candidate program(s) 106. In some examples, the candidate program analyzer 114 uses a model generated via neural network training to select a particular fitness function from a collection of possible fitness functions to evaluate the candidate program(s) 106. One or more or, in some examples, all, of the aforementioned neural network models (i.e., program length prediction, fitness function generation, fitness function selection) can be implemented in connection with the example genetic algorithm programmer 102 of FIG. 1A.

Artificial intelligence (AI), including machine learning (ML), deep learning (DL), and/or other artificial machine-driven logic, enables machines (e.g., computers, logic circuits, etc.) to use a model to process input data to generate an output based on patterns and/or associations previously learned by the model via a training process. For instance, the model may be trained with data to recognize patterns and/or associations and follow such patterns and/or associations when processing input data such that other input(s) result in output(s) consistent with the recognized patterns and/or associations.

Many different types of machine learning models and/or machine learning architectures exist. In examples disclosed herein, deep neural network models are used. In general, machine learning models/architectures that are suitable to use in the example approaches disclosed herein will be based on supervised learning. However, other types of machine learning models could additionally or alternatively be used such as, for example, unsupervised learning.

As noted above, ML/AI models are trained to produce a particular output given a particular input. Different types of training may be performed, based on the type of ML/AI model and/or the expected output. For example, supervised training uses inputs and corresponding expected outputs to iterate over combinations of select parameters the ML/AI model that reduce an amount of error.

In general, implementing a ML/AI system involves two phases, a learning/training phase and an inference phase. In the learning/training phase, a training algorithm is used to train a model to operate in accordance with patterns and/or associations based on, for example, training data. In general, the model includes internal parameters that guide how input data is transformed into output data, such as through a series of nodes and connections within the model to transform input data into output data. Additionally, hyperparameters are used as part of the training process to control how the learning is performed (e.g., a learning rate, a number of layers to be used in the machine learning model, etc.). Hyperparameters are defined to be training parameters that are determined prior to initiating the training process.

Different types of training may be performed based on the type of ML/AI model and/or the expected output. For example, supervised training uses inputs and corresponding expected (e.g., labeled) outputs to select parameters (e.g., by iterating over combinations of select parameters) for the ML/AI model that reduce model error. As used herein, labelling refers to an expected output of the machine learning model (e.g., a classification, an expected output value, etc.). Alternatively, unsupervised training (e.g., used in deep learning, a subset of machine learning, etc.) involves inferring patterns from inputs to select parameters for the ML/AI model (e.g., without the benefit of expected (e.g., labeled) outputs).

In examples disclosed herein, ML/AI models are trained using training algorithms such as a categorical cross-entropy optimization model or stochastic gradient descent. However, any other training algorithm may additionally or alternatively be used. In examples disclosed herein, training can be performed based on early stopping principles in which training continues until the model(s) stop improving. In examples disclosed herein, training can be performed remotely or locally. In some examples, training may initially be performed remotely. Further training (e.g., retraining) may be performed locally based on data generated as a result of execution of the models. Training is performed using hyperparameters that control how the learning is performed (e.g., a learning rate, a number of layers to be used in the machine learning model, etc.). For example, some example neural networks disclosed herein can have three hidden layers. However, neural networks having other numbers of layers can be used. In some examples, the neural networks are classification neural networks. In other examples, the neural networks may be regression neural networks (e.g., to predict program length). In some examples re-training may be performed. Such re-training may be performed in response to the identification of one or more effective end result programs as a result of the genetic programming process.

Training is performed using training data. In examples disclosed herein, the training data originates from previously generated programs (e.g., created for operational use) and/or randomly generated programs generated for the purposes of training. Because supervised training is used, the training data is labeled. In example disclosed herein, labeling is applied to training data based on, for example, the number of errors introduced into a program during the generation of the training data, program length, etc. In some examples, the training data is pre-processed. For example, when the training data is based on randomly generated programs, certain ones of the random programs that output the same result may be filtered or removed from the training data to avoid biasing the training data. In some examples, the training data is sub-divided such that a portion of the data (e.g., 20%) is used for validation purposes.

Once training is complete, the model(s) are stored in one or more databases (e.g., databases 136, 152, 164 of FIG. 1A). One or more of the models may then be executed by, for example, the artificial genetic algorithm programmer 102 as disclosed below.

Once trained, the deployed model may be operated in an inference phase to process data. In the inference phase, data to be analyzed (e.g., live data) is input to the model, and the model executes to create an output. This inference phase can be thought of as the AI “thinking” to generate the output based on what it learned from the training (e.g., by executing the model to apply the learned patterns and/or associations to the live data). In some examples, input data undergoes pre-processing before being used as an input to the machine learning model. Moreover, in some examples, the output data may undergo post-processing after it is generated by the AI model to transform the output into a useful result (e.g., a display of data, an instruction to be executed by a machine, etc.).

In some examples, output of the deployed model may be captured and provided as feedback. By analyzing the feedback, an accuracy of the deployed model can be determined. If the feedback indicates that the accuracy of the deployed model is less than a threshold or other criterion, training of an updated model can be triggered using the feedback and an updated training data set, hyperparameters, etc., to generate an updated, deployed model.

The example system of FIG. 1A includes a first computing system 121 for training a neural network to predict a length of a program (e.g., number of executable statements). The example first computing system 121 includes a first neural network processor 122. In examples disclosed herein, the first neural network processor 122 implements a first neural network.

The example first computing system 121 of FIG. 1A includes a first neural network trainer 126. The example first neural network trainer 126 of FIG. 1A performs training of the neural network implemented by the first neural network processor 122. In some examples disclosed herein, training is performed using a categorical cross-entropy optimization algorithm. However, other approaches to training a neural network may additionally or alternatively be used.

The example first computing system 121 of FIG. 1A includes a first training controller 128. The example training controller 128 instructs the first neural network trainer 126 to perform training of the neural network based on first training data 130. In the example of FIG. 1A, the first training data 130 used by the first neural network trainer 126 to train the neural network is stored in a database 132. The example database 132 of the illustrated example of FIG. 1 A is implemented by any memory, storage device and/or storage disc for storing data such as, for example, flash memory, magnetic media, optical media, etc. Furthermore, the data stored in the example database 132 may be in any data format such as, for example, binary data, comma delimited data, tab delimited data, structured query language (SQL) structures, image data, etc. While in the illustrated example database 132 is illustrated as a single element, the database 132 and/or any other data storage elements described herein may be implemented by any number and/or type(s) of memories.

In the example of FIG. 1A, the training data 130 can include known programs and/or programs that have been randomly (e.g., pseudo-randomly) generated for purposes of training. In some examples, the training data includes programs generated by, for instance, the genetic program generator 116 and determined to be effective end result program(s) by the candidate program analyzer 114 (i.e., the end result program(s) 118 that satisfy the input/output data). The training data 130 is labeled with the length of the respective programs in the training data (e.g., the number of statements). The first neural network trainer 126 trains the neural network implemented by the neural network processor 122 by running the programs using the training data 130. In some examples, the programs are run offline to generate the input/output pairs and the training data 130 does not include the programs, but instead includes only the inputs, the outputs, and number of lines of executable code for each program. Based on the input/output data and the respective program lengths in the training data 130, the first neural network trainer 126 trains the neural network 122 to predict the lengths of the programs based on the input/output pairs.

A program length prediction model 134 is generated as a result of the neural network training. The program length prediction model 134 is stored in a database 136. The databases 132, 136 may be the same storage device or different storage devices.

In the example of FIG. 1A, the program length prediction model 134 is executed by the program length predictor 120 of the artificial genetic algorithm programmer 102. In particular, the program length predictor 120 executes the program length prediction model 134 in view of the target input and output data 108, 110 (which may be different from the input/output data of the training data 130) to determine a predicted program length of an effective end result program that satisfies the input/output data 108, 110. As illustrated in FIG. 1A, the program length predictor 120 is in communication with the random program generator 104 of the example genetic algorithm programmer 102. The random program generator 104 generates candidate program(s) 106 having the predicted program length as determined by the program length predictor 120 (e.g., approximately 4-7 lines of executable instructions). In some examples, all of the programs generated by the random program generator 104 have the same predicted length. In some other examples, the program length predictor 120 determines a program length distribution (e.g., mean, standard deviation) and the random program generator 104 generates programs having respective lengths based on the distribution. As a result, the candidate program(s) 106 generated by the random program generator 104 and, thus, the end result program(s) generated by the genetic program generator 116 via breeding of the candidate program(s) 106, are more likely to have program lengths at or near the predicted program length of an effective end result program. Therefore, the genetic algorithm programmer 102 can more efficiently generate effective end result program(s) based on random program(s) that are initialized based on the predicted program length.

In the example of FIG. 1A, the effective end result program(s) 118 that generate a correct output for each input in view of the target input/output data 108, 110 can be added to the training data 130 and used to retrain the neural network implemented by the first neural network processor 122. In some examples, the neural network can be tuned to predict program length for a particular data domain or data type (e.g., financial data) by retraining based on the effective end result program(s) 118.

The example system of FIG. 1A includes a second computing system 137 for training a neural network to generate a fitness function based on machine learning. The example second computing system 137 includes a second neural network processor 138. In examples disclosed herein, the second neural network processor 138 implements a second neural network different from the first neural network implemented by the first neural network processor 122.

The example second computing system 137 of FIG. 1A includes a second neural network trainer 140. The example second neural network trainer 140 of FIG. 1A performs training of the second neural network implemented by the second neural network processor 138. In some examples disclosed herein, training is performed using a categorical cross-entropy optimization algorithm. However, other approach to training a neural network may additionally or alternatively be used.

The example second computing system 137 of FIG. 1A includes a second training controller 142. The example training controller 142 instructs the second neural network trainer 140 to perform training of the neural network based on second training data 144. In the example of FIG. 1A, the second training data 144 used by the second neural network trainer 140 to train the neural network is stored in a database 146. The example database 146 of the illustrated example of FIG. 1A is implemented by any memory, storage device and/or storage disc for storing data such as, for example, flash memory, magnetic media, optical media, etc. Furthermore, the data stored in the example database 146 may be in any data format such as, for example, binary data, comma delimited data, tab delimited data, structured query language (SQL) structures, image data, etc. While in the illustrated example database 146 is illustrated as a single element, the database 146 and/or any other data storage elements described herein may be implemented by any number and/or type(s) of memories.

In the example of FIG. 1A, the training data 144 can include known programs collected from prior uses and/or randomly generated for purposes of training. In some examples, the training data 144 includes the effective end result program(s) 118 generated by the genetic program generator 116. In some examples, the programs are run offline to generate the input/output pairs and the training data 144 does not include the programs, but instead includes only the inputs, the outputs, and fitness or correctness score(s) indicating that the data was either generated by a program containing no errors or a program containing a particular number of errors.

The example second computing system 137 of FIG. 1A includes a program mutator 148. In this example, the mutator 148 implements means for introducing error(s) into a program. In the example of FIG. 1A, the mutator 148 creates copies of known program(s), where each copy of a respective program includes one or more modifications that create errors in the program such that the program may no longer produce the correct output for a given input. Example error(s) that can be introduced into a program include a deletion error, in which a random statement is deleted from the program; an insertion error, in which a new statement is inserted into a random position in the program; and/or a change error, in which a random statement is deleted from the program and a new statement is added at the position where the deletion occurred. The mutated programs are run for a set of inputs (e.g., the same inputs for which the program(s) that do not include error(s) were run) to generate outputs. The inputs and the outputs generated by the mutated programs are stored in the training data 144 and associated with a fitness or correctness score indicating that the data was generated by program(s) containing errors (e.g., one error, two errors).

The second neural network trainer 140 trains the neural network implemented by the second neural network processor 138 based on the training data 144, including the inputs, the correct outputs generated by the program(s) without error(s), the outputs generated by the mutated program(s), and the fitness or correctness scores. As a result of the training, a fitness function model 150 is generated. The fitness function model 150 is stored in a database 152. The databases 146, 152 may be the same storage device or different storage devices.

In the example of FIG. 1A, the candidate program analyzer 114 uses the fitness function model 150 generated during the training to assess the fitness of the candidate program(s) 106 generated by the random program generator 104. In particular, the candidate program analyzer 114 executes the fitness function model 150 to analyze the candidate program(s) 106 based on the input data 108, the correct or target output data 110, and the output data generated by the candidate program(s) 106 being evaluated. The candidate program analyzer 114 determines a correctness score for each candidate program 106 indicative of the number of errors in the respective candidate programs 106 based on the fitness function model 150. In some examples, the candidate program analyzer 114 executes the fitness function model 150 for multiple input/output examples 108, 110 for a particular candidate program 106. The correctness scores corresponding to each input/output example are combined (e.g., averaged) to create a correctness estimate for the particular candidate program 106. In such examples, the candidate program analyzer 114 uses the correctness estimate to select the candidate program(s) 106 that are determined to be more correct or effective at providing a desired output than other candidate programs based on the fitness function model 150 for breeding by the genetic program generator 116. The candidate program analyzer 114 executes the fitness function model 150 to assess each candidate program 106 to determine a correctness score (or correctness estimate based on two or more correctness scores) for each candidate program 106.

In the example of FIG. 1A, the effective end result program(s) 118 produced by the genetic program generator 116 can be added to the training data 144 and used to retrain the neural network implemented by the second neural network processor 138. In some examples, the neural network can be tuned to generate or adjust the fitness function model 150 for a particular data domain or data type (e.g., financial data) by retraining based on the effective end result program(s) 118.

In some examples, use of a neural network-based fitness function such as the fitness function model 150 of FIG. 1A to evaluate a candidate program results in the identification of an effective end result program approximately 90% of the time. In some such examples, evaluations of the candidate program using the Manhattan Distance results in identification of an effective end result program approximately 30% of the time. Further, the number of generations (e.g., the number of breeding iterations) to find an effective end result program decreased from 4,500 using Manhattan Distance to 780 with the neural network-based fitness function. Such a decrease represents a significant reduction in time and processing cycles/computing resource usage. Thus, in some examples, the use of a fitness function model that is derived based on neural network training as disclosed herein can improve efficiency and accuracy in identifying an effective end result program.

The example system of FIG. 1A includes a third computing system 153 for training a neural network to select a fitness function from a collection of possible known fitness functions to evaluate a candidate program 106. The example third computing system 153 includes a third neural network processor 154. In examples disclosed herein, the third neural network processor 154 implements a third neural network different from the first neural network implemented by the first neural network processor 122 or the second neural network implemented by the second neural network processor 138.

The example third computing system 153 of FIG. 1A includes a third neural network trainer 156. The example third neural network trainer 156 of FIG. 1 A performs training of the neural network implemented by the third neural network processor 154. In some examples disclosed herein, training is performed using a categorical cross-entropy optimization algorithm. However, other approaches to training a neural network may additionally or alternatively be used.

The example third computing system 153 of FIG. 1A includes a third training controller 158. The example training controller 158 instructs the third neural network trainer 156 to perform training of the neural network based on third training data 159. In the example of FIG. 1A, the training data 159 used by the third neural network trainer 156 to train the neural network is stored in a database 160. The example database 160 of the illustrated example of FIG. 1A is implemented by any memory, storage device and/or storage disc for storing data such as, for example, flash memory, magnetic media, optical media, etc. Furthermore, the data stored in the example database 160 may be in any data format such as, for example, binary data, comma delimited data, tab delimited data, structured query language (SQL) structures, image data, etc. While in the illustrated example database 160 is illustrated as a single element, the database 160 and/or any other data storage elements described herein may be implemented by any number and/or type(s) of memories.

In the example of FIG. 1A, the training data 159 can include a plurality of fitness functions, such as fitness functions based on distance metrics (e.g., Manhattan distance) and fitness functions generated based on machine learning (e.g., the fitness function model 150). The training data 159 also includes sample programs and corresponding input/output pairs. In the example of FIG. 1A, the input/output pairs in the training data 159 are associated with a particular fitness function that outperforms other fitness functions for each input/output example. In some examples, the programs are run offline to generate the input/output pairs and the training data 159 does not include the programs, but instead includes only the inputs, the outputs, and a particular fitness function for a respective input/output pair. The third neural network trainer 156 trains the neural network implemented by the neural network processor 154 to select the optimal fitness function for the input/output data based on the training data 159.

A fitness function selection model 162 is generated as result of the neural network training. The fitness function selection model 162 is stored in a database 164. The databases 160, 164 may be the same storage device or different storage devices

In the example of FIG. 1A, the candidate program analyzer 114 executes the fitness function selection model 162 based on the input/output data 108, 110 to select the appropriate (e.g., optimal fitness function) for evaluating a particular candidate program 106 from a collection of possible known fitness functions. The candidate program analyzer 114 uses the selected fitness function (e.g., the fitness function model 150, Manhattan distance) to determine the fitness of a particular candidate program 106 and to identify whether the particular candidate program 106 should be used for breeding.

As shown in FIG. 1A, the effective end result program(s) 118 generated by the genetic program generator 116 can be provided as training data 159 for training the neural network implemented by the third neural network processor 154. In the example of FIG. 1A, when being added to the training data 159, the effective end result program(s) 118 are run through the genetic algorithm programmer 102 with each fitness function in the training data 159 to determine the optimal fitness function for the effective end result program(s) 118. The effective end result program(s) 118 and the corresponding optimal fitness function data for the effective end result program(s) 118 can be added to the training data 159.

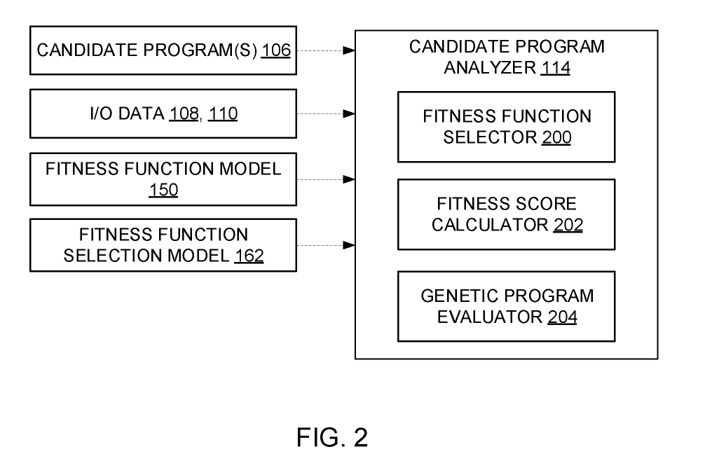

FIG. 2 is a block diagram of an example implementation of the example candidate program analyzer 114 of FIG. 1A. As mentioned above, the example candidate program analyzer 114 is constructed to evaluate the candidate program(s) 106 generated by the example random program generator 104 of FIG. 1A to assess the fitness of the candidate program(s) for use in breeding operation(s) performed by the genetic program generator 116 (e.g., mutation, crossover). The example candidate program analyzer 114 is also constructed to evaluate the end result programs generated by the example genetic program generator 116 of FIG. 1 A to determine if the end result programs satisfy the target input/output criteria and, thus, can be considered effective end result programs. In the example of FIG. 2, the candidate program analyzer 114 is implemented by one or more processors.

The example candidate program analyzer 114 of FIG. 2 includes a fitness function selector 200. In this example, the fitness function selector 200 implements means for executing the fitness function selection model 162. In the example of FIG. 2, the fitness function selector 200 executes the fitness function selection model 162 generated by the neural network implemented by the third neural network processor 154 of FIG. 1A. In particular, the example fitness function selector 200 of FIG. 2 executes the fitness function selection model 162 to select an appropriate (e.g., optimal) fitness function for a given program from a collection of possible fitness functions and in view of, for instance, the target input/output data 108, 110. The fitness function selected by the fitness function selector 200 can include a neural network-based fitness function (e.g., the fitness function model 150), a distance-based fitness function such as Manhattan distance, or any other type of fitness function.

The example candidate program analyzer 114 includes a fitness score calculator 202. In this example, the fitness score calculator 202 implements means for calculating fitness score(s) of candidate program(s) 106. In the example of FIG. 2, the example fitness function selector 200 communicates the selected fitness function to the fitness score calculator 202. The example fitness score calculator 202 executes the selected fitness function to analyze the candidate program(s) 106 in view of the target input/output data 108, 110.

In some examples, the fitness score calculator 202 executes the fitness function model 150 generated during training of the neural network implemented by the second neural network processor 138 of FIG. 2 to assess the fitness of the candidate program(s) 106 generated by the random program generator 104. In some examples, the fitness score calculator 202 considers multiple input/output examples 108, 110 when executing the fitness function model 150 for a particular candidate program 106. In such examples, the fitness score calculator 202 combines (e.g., averages) the fitness (e.g., correctness) scores for the candidate program 106 being evaluated to determine a correctness estimate for the candidate program 106.

Based on the correctness score (or the correctness estimate based on multiple correctness scores), the fitness score calculator 202 determines which candidate programs 106 should be associated with a higher probability of being used for breeding over other candidate programs 106. The example fitness score calculator 202 of FIG. 2 communicates with the example genetic program generator 116 of FIG. 1A (e.g., via one or more wired or wireless connections) to identity the candidate program(s) 106 that should have a higher probability of being randomly selected for genetic programming by the genetic program generator 116 over other candidate program(s) 106 (e.g., candidate program(s) 106 with lower performing fitness scores). In some examples, the fitness score calculator 202 removes or filters out the candidate programs that do not satisfy fitness score threshold(s) (e.g., predefined thresholds) and those programs are not used by the genetic program generator 116. Thus, the fitness score calculator 202 generates a biased distribution of the candidate program(s) 106 for selection by the genetic program generator 116 for use in the breeding operation(s).

The example candidate program analyzer 114 of FIG. 2 includes a genetic program evaluator 204. In this example, the genetic program evaluator 204 implements means for evaluating the end result program(s). The example genetic program evaluator 204 analyzes the end result program(s) 118 (e.g., the evolved candidate program(s)) generated by the genetic program generator 116 to determine if the end result program(s) are solutions to the target input/output criteria 108, 110. For example, the genetic program evaluator 204 determines if a particular end result program satisfies all of the input/output examples 108, 110. Based on the analysis, the genetic program evaluator 204 determines if the particular end result program is an effective end result program 118. If none of the end result program(s) 118 are identified by the genetic program evaluator 204 as being effective end result program(s), then the genetic program evaluator 204 may determine that further iterations of the genetic breeding should be performed (e.g., if an iteration threshold has not yet been met). The additional iterations of the genetic breeding can be performed using, for example, at least some of the evolved candidate program(s) generated during earlier iterations of the genetic algorithm.

While an example manner of implementing the genetic algorithm programmer 102 is illustrated in FIGS. 1 and 2, one or more of the elements, processes and/or devices illustrated in FIGS. 1 and/or 2 may be combined, divided, re-arranged, omitted, eliminated and/or implemented in any other way. Further, the example random program generator 104, the example database 112, the example candidate program analyzer 114, the example genetic program generator 116, the example program length predictor 120, the example fitness function selector 200, the example fitness score calculator 202, the example genetic program evaluator 204 and/or, more generally, the example genetic algorithm programmer 102 of FIGS. 1 and 2 may be implemented by hardware, software, firmware and/or any combination of hardware, software and/or firmware. Thus, for example, any of the example random program generator 104, the example database 112, the example candidate program analyzer 114, the example genetic program generator 116, the example program length predictor 120, the example fitness function selector 200, the example fitness score calculator 202, the example genetic program evaluator 204, and/or, more generally, the example genetic algorithm programmer 102 of FIGS. 1 and 2 could be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example random program generator 104, the example database 112, the example candidate program analyzer 114, the example genetic program generator 116, the example program length predictor 120, the example fitness function selector 200, the example fitness score calculator 202, and/or the example genetic program evaluator 204 is/are hereby expressly defined to include a non-transitory computer readable storage device or storage disk such as a memory, a digital versatile disk (DVD), a compact disk (CD), a Blu-ray disk, etc. including the software and/or firmware. Further still, the example genetic algorithm programmer 102 of FIGS. 1 and 2 may include one or more elements, processes and/or devices in addition to, or instead of, those illustrated in FIGS. 1 and/or 2, and/or may include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

While an example manner of implementing the first computing system 121 is illustrated in FIG. 1A, one or more of the elements, processes and/or devices illustrated in FIG. 1A may be combined, divided, re-arranged, omitted, eliminated and/or implemented in any other way. Further, the example neural network processor 122, the example trainer 126, the example training controller 128, the example database 132, and/or, more generally, the example first computing system 121 of FIG. 1A may be implemented by hardware, software, firmware and/or any combination of hardware, software and/or firmware. Thus, for example, any of the example neural network processor 122, the example trainer 126, the example training controller 128, the example database 132, and/or, more generally, the example first computing system 121 of FIG. 1A could be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example neural network processor 122, the example trainer 126, the example training controller 128, and/or the example database 132 is/are hereby expressly defined to include a non-transitory computer readable storage device or storage disk such as a memory, a digital versatile disk (DVD), a compact disk (CD), a Blu-ray disk, etc. including the software and/or firmware. Further still, the example first computing system 121 may include one or more elements, processes and/or devices in addition to, or instead of, those illustrated in FIG. 1A, and/or may include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

While an example manner of implementing the second computing system 137 is illustrated in FIG. 1A, one or more of the elements, processes and/or devices illustrated in FIG. 1A may be combined, divided, re-arranged, omitted, eliminated and/or implemented in any other way. Further, the example neural network processor 138, the example trainer 140, the example training controller 142, the example database 146, the example mutator 148, and/or, more generally, the example second computing system 137 of FIG. 1A may be implemented by hardware, software, firmware and/or any combination of hardware, software and/or firmware. Thus, for example, any of the example neural network processor 138, the example trainer 140, the example training controller 142, the example database 146, the example mutator 148, and/or, more generally, the example second computing system 137 of FIG. 1A could be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example neural network processor 138, the example trainer 140, the example training controller 142, the example database 146, and/or the example mutator 148 is/are hereby expressly defined to include a non-transitory computer readable storage device or storage disk such as a memory, a digital versatile disk (DVD), a compact disk (CD), a Blu-ray disk, etc. including the software and/or firmware. Further still, the example second computing system 137 may include one or more elements, processes and/or devices in addition to, or instead of, those illustrated in FIG. 1A, and/or may include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

While an example manner of implementing the third computing system 153 is illustrated in FIG. 1A, one or more of the elements, processes and/or devices illustrated in FIG. 1A may be combined, divided, re-arranged, omitted, eliminated and/or implemented in any other way. Further, the example neural network processor 154, the example trainer 156, the example training controller 158, the example database 160, and/or, more generally, the example third computing system 153 of FIG. 1A may be implemented by hardware, software, firmware and/or any combination of hardware, software and/or firmware. Thus, for example, any of the example neural network processor 154, the example trainer 156, the example training controller 158, the example database 160, and/or, more generally, the example third computing system 153 of FIG. 1A could be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example neural network processor 154, the example trainer 156, the example training controller 158, and/or the example database 160 is/are hereby expressly defined to include a non-transitory computer readable storage device or storage disk such as a memory, a digital versatile disk (DVD), a compact disk (CD), a Blu-ray disk, etc. including the software and/or firmware. Further still, the example third computing system 153 may include one or more elements, processes and/or devices in addition to, or instead of, those illustrated in FIG. 1A, and/or may include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

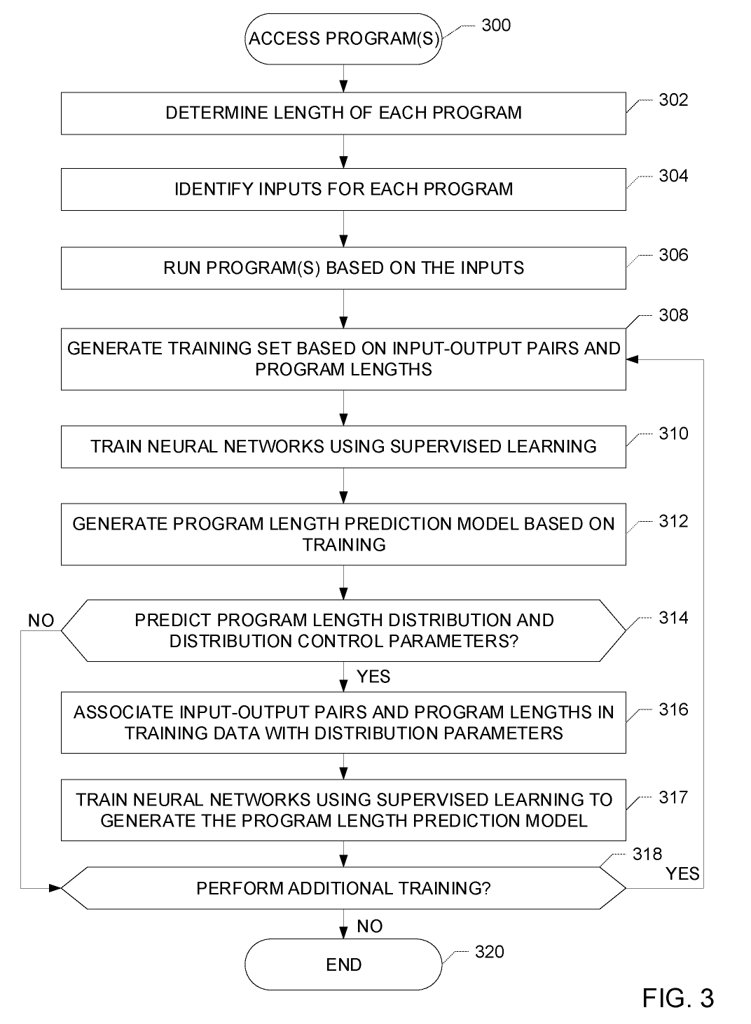

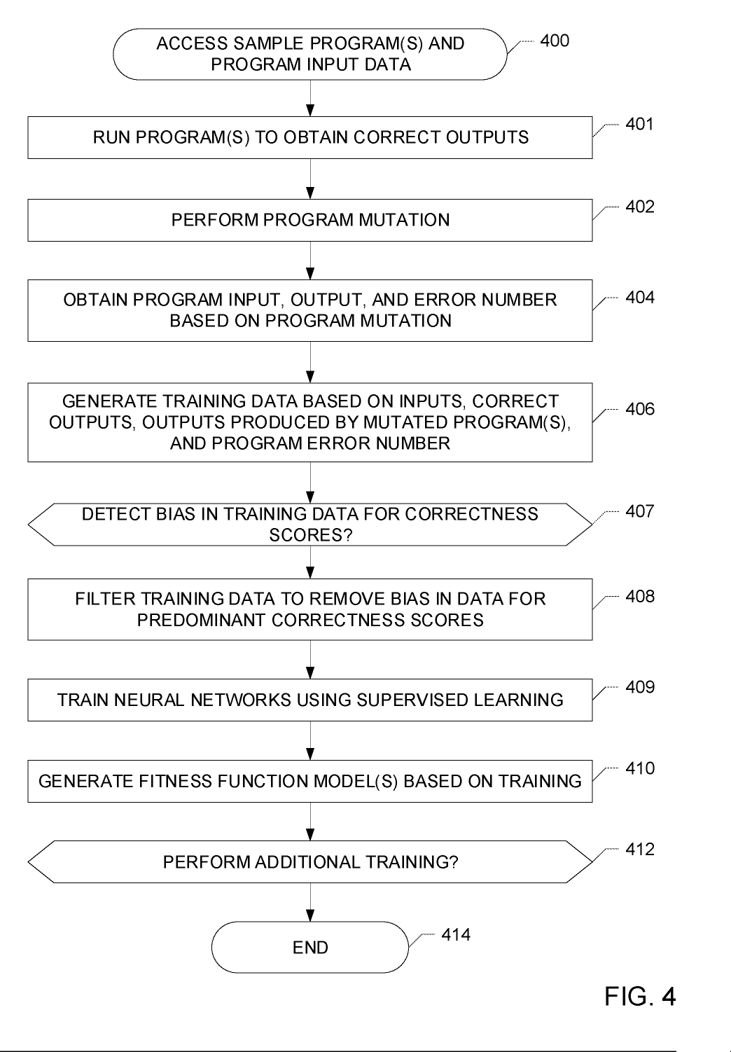

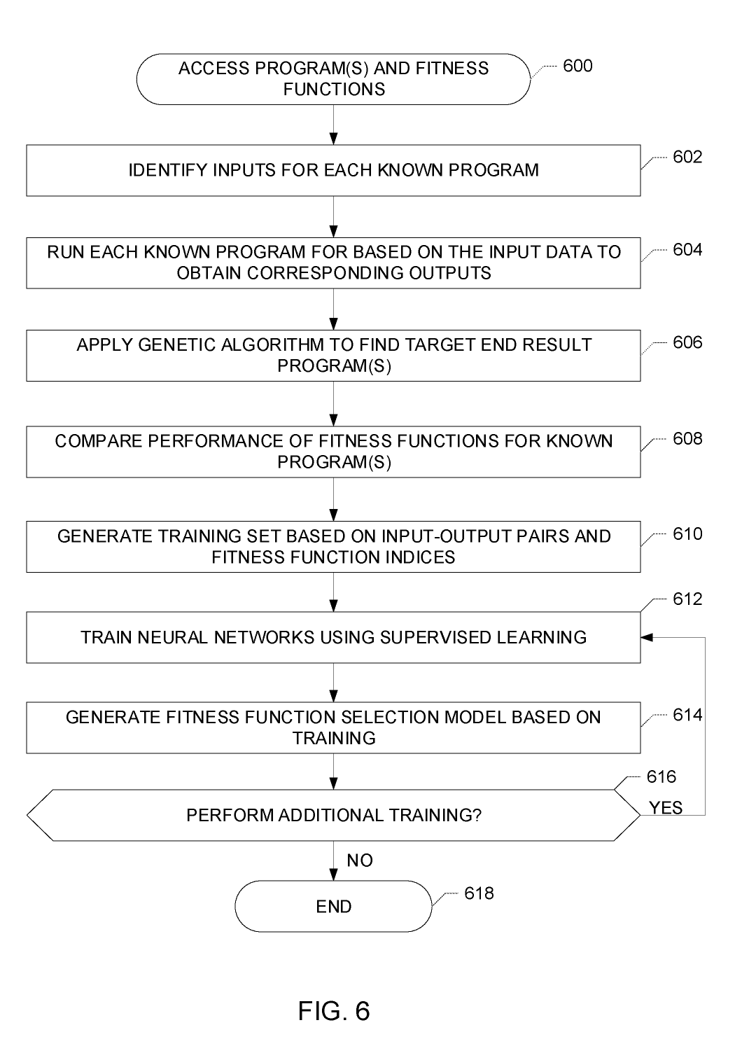

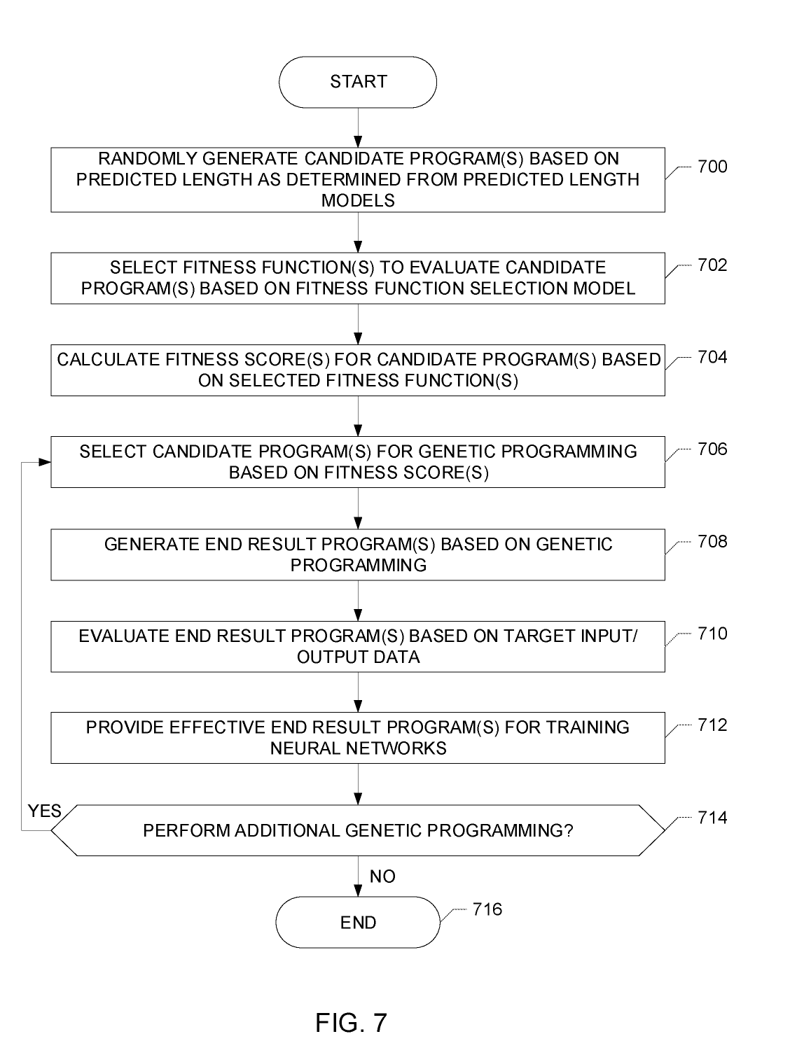

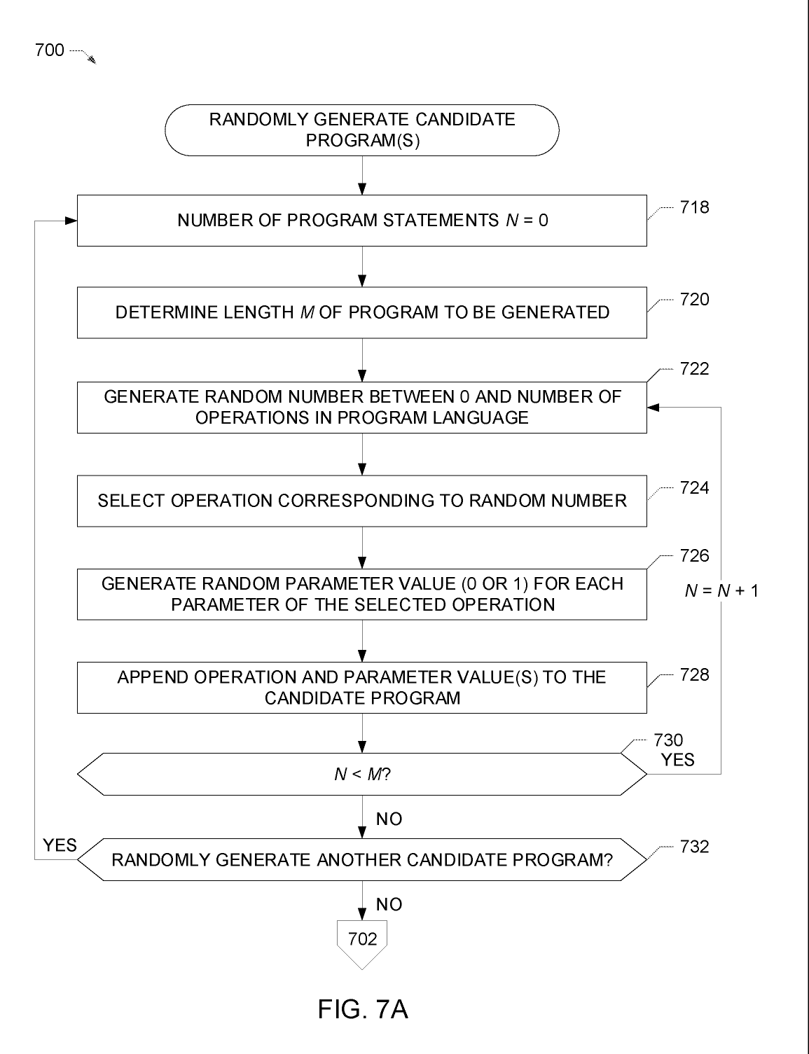

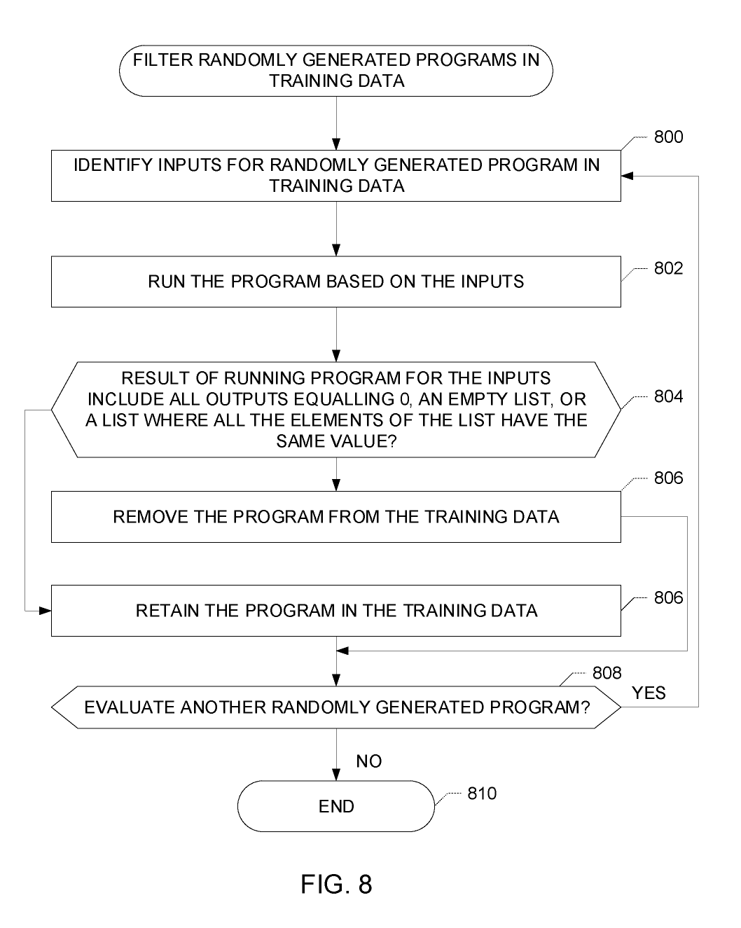

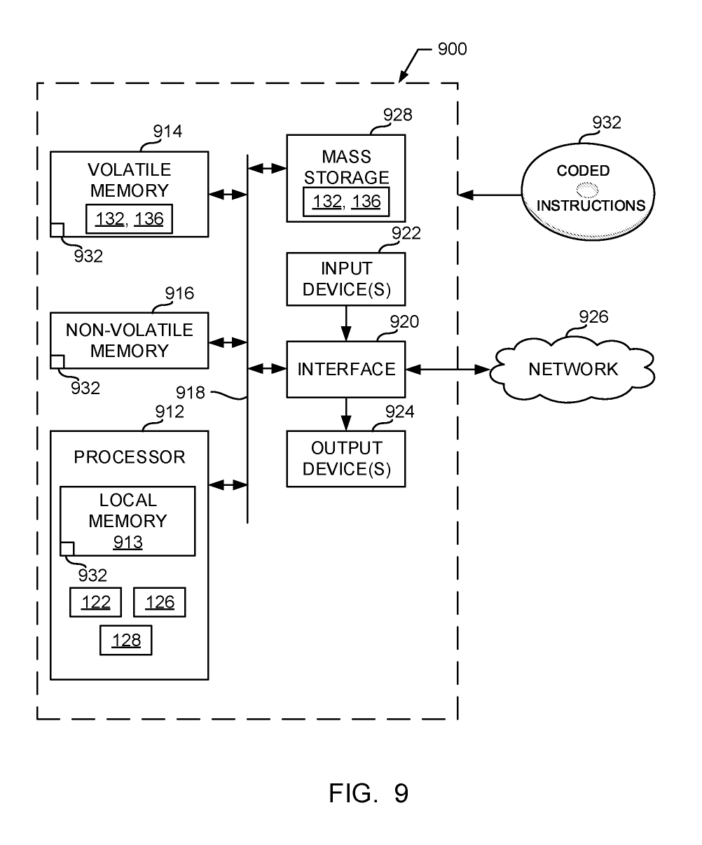

A flowchart representative of example hardware logic, machine readable instructions, hardware implemented state machines, and/or any combination thereof for implementing the example first computing system 121 of FIG. 1A is shown in FIGS. 3 and 8. Flowcharts representative of example hardware logic, machine readable instructions, hardware implemented state machines, and/or any combination thereof for implementing the example second computing system 137 of FIG. 1A is shown in FIGS. 4, 5, 5A, and 8. A flowchart representative of example hardware logic, machine readable instructions, hardware implemented state machines, and/or any combination thereof for implementing the example third computing system 153 of FIG. 1A is shown in FIGS. 6 and 8. A flowchart representative of example hardware logic, machine readable instructions, hardware implemented state machines, and/or any combination thereof for implementing the example genetic algorithm programmer 102 of FIG. 1A is shown in FIGS. 7 and 7A. The machine readable instructions of FIGS. 3-8 may be one or more executable programs or portion(s) of an executable program for execution by a computer processor such as the processors 912, 1012, 1112, 1212 shown in the example processor platforms 900, 1000, 1100, 1200 discussed below in connection with FIGS. 9-12. The program(s) may be embodied in software stored on a non-transitory computer readable storage medium such as a CD-ROM, a floppy disk, a hard drive, a DVD, a Blu-ray disk, or a memory associated with the processor(s) 912, 1012, 1112, 1212 but the entire program(s) and/or parts thereof could alternatively be executed by a device other than the processor(s) 912, 1012, 1112, 1212 and/or embodied in firmware or dedicated hardware. Further, although the example program(s) are described with reference to the flowcharts illustrated in FIGS. 3-8, many other methods of implementing the example first computing system 121, the example second computing system 137, the example third computing system 153, and/or the example genetic algorithm programmer 102 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine readable instructions described herein may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine readable instructions may be fragmented and stored on one or more storage devices and/or computing devices (e.g., servers). The machine readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine readable instructions may be stored in multiple parts, which are individually compressed, encrypted, and stored on separate computing devices, wherein the parts when decrypted, decompressed, and combined form a set of executable instructions that implement a program such as that described herein.

In another example, the machine readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, the disclosed machine readable instructions and/or corresponding program(s) are intended to encompass such machine readable instructions and/or program(s) regardless of the particular format or state of the machine readable instructions and/or program(s) when stored or otherwise at rest or in transit.

The machine readable instructions described herein can be implemented by any past, present, or future programming language. For example, the machine readable instructions may be implemented using any of the following languages: C, C++, Java, C#, Perl, Python, JavaScript, HyperText Markup Language (HTML), Structured Query Language (SQL), Swift, etc.

As mentioned above, the example processes of FIGS. 3-8 may be implemented using executable instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc. may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, and (3) at least one A and at least one B.

FIG. 3 is a flowchart representative of example machine readable instructions which, when executed by the example first computing system 121 of FIG. 1A, cause the example first computing system 121 to train a neural network to predict a length of a program (e.g., number of statements of executable instructions). The example instructions of FIG. 3, when executed by the first computing system 121 of FIG. 1A, results in a neural network and/or a model thereof, that can be distributed to other computing systems, such as the program length predictor 120 of FIG. 1A.

The example of FIG. 3 begins with the training controller 128 accessing programs stored in the example database 132 (block 300). The program data can include known programs from prior uses and/or or randomly generated programs for purposes of training. In some examples, the programs include end result program(s) 118 identified by the candidate program analyzer 114 as being effective program(s) in that the programs correctly produce target outputs for known inputs.

The example training controller 128 of FIG. 1 A determines the length of each known program by counting, for example, the number of statements of executable instructions in each program (block 302). Data indicating the length of each program can be stored in the database 132.

The example training controller 128 identifies inputs for each program (block 304). The inputs can be known inputs (e.g., user-defined). The example training controller 128 runs each the program based on the inputs to generate corresponding outputs (block 306). The input/output pairs can be stored in the database 132.

In the example of FIG. 3, the training controller 128 generates the training data 130 based on the input/output pairs and the program lengths (block 308). The input/output pairs are labeled in the training data 130 with the length of the program that generated the input/output pairs.

The example training controller 128 instructs the neural network trainer 126 to perform training of the neural network 122 using the training data 130 (block 310). In the example of FIG. 3, the training is based on supervised learning. As a result of the training, the program length prediction model 134 is generated (block 312). Based on the program length prediction model, the neural network is trained to predict a length of a program that provides correct output(s) for given input(s). The program length prediction model 134 can be stored in the database 136 for access by the program length predictor 120 of the genetic algorithm programmer 102.

In some examples, the random program generator 104 generates random programs having the same length based on the predicted length. In other examples, the random program generator 104 generates programs having a particular length based on a program length distribution as predicted by the neural network. In such instances, the example instructions of FIG. 3 include predicting a program length distribution and control parameters (e.g., mean, standard deviation of the predicted length) as part of training the neural network (block 314).

In examples where the neural network is trained to predict the program length distribution, the training controller 128 associates or maps the input/output pairs that were generated when the known programs were run (i.e., at block 306) and the corresponding known program lengths with a particular type of distribution (e.g., a uniform distribution, a normal distribution, a Poisson distribution, etc.) and distribution control parameter(s) (e.g., mean, standard deviation, etc.) and uses the mapping data to train the neural network to generate the program length prediction model that can be used to predict program length distribution (block 316, 317). In such examples, the program length predictor 120 executes the program length prediction model 134 to predict a program length distribution for candidate programs 106 that will result in the genetic algorithm programmer 102 producing effective end result program(s) based on the candidate programs 16. The random program generator 104 generates the candidate programs 106 having lengths based on the program length distribution. In some examples, the instructions of FIG. 3 train the neural network to predict the program length distribution after generating the training data at block 308 instead of (e.g., initially) training the model to predict the same program length for all programs.

The example instructions of FIG. 3 end when no additional training (e.g., retraining) is to be performed (blocks 318, 320).

FIG. 4 is a flowchart representative of example machine readable instructions which, when executed by the example second computing system 137 of FIG. 1A, cause the example second computing system 137 to train a neural network such as the neural network implemented by the second neural network processor 138 to generate a neural-network based fitness function model. The example instructions of FIG. 4, when executed by the second computing system 137 of FIG. 1A, results in a neural network and/or a model thereof, that can be distributed to other computing systems, such as the genetic algorithm programmer 102 of FIG. 1A.

In the example of FIG. 4, the second training controller 142 accesses sample programs that are known to produce correct outputs for given inputs (block 400). The example training controller 142 runs the known programs based on known input data to determine corresponding known outputs (block 401).

In the example of FIG. 4, for each program, the mutator 148 of FIG. 1A mutates (e.g., modifies) the known programs by introducing error(s) into the program (block 402). The example second training controller 142 of FIG. 1A runs the programs including the mutation(s) to obtain the input/output pairs generated by the respective programs containing the errors (block 404).

In the example of FIG. 4, the second training controller 142 generates the training data 144 based on the inputs, the correct outputs associated with each input as determined by the programs that do not contain any errors, the outputs determined by the mutated programs based on the inputs, and the number of errors in the mutated programs (block 406). The training data 144 includes input/output pairs labeled with the number of errors introduced into the programs that generated the input/output pairs. For example, the input/output pairs that were generated from programs without any errors are labeled with an indicator that those pairs do not contain any errors. For input/output pairs generated from a program with two errors, the input/output pairs are labeled to indicate that those pairs were generated from a program with two errors. The example training data 144 can include other data associated with programs that could affect the effectiveness (e.g., correctness) of the programs and, thus, be of interest as inputs when in applying the neural network model, such as functions called or used by a program.

In some examples, the mutation process, which is performed for each known program, can result in training data associated with an unequal representation of fitness or correctness scores for the programs, which can create bias in the resulting neural network model generated from the training data with respect to the predominant correctness score (block 407). In some examples of FIG. 4, the training controller 142 filters the training data to remove data associated with the predominant correctness score(s) (block 408). As a result, the filtered training data includes substantially equally represented fitness or correctness scores.

In the example of FIG. 4, the example training controller 142 instructs the neural network trainer 140 to perform training of the neural network 138 using the training data 144 (e.g., the filtered training data) (block 409). In the example of FIG. 4, the training is based on supervised learning. As a result of the training, the neural network is trained to predict the number of errors in a program as represented by the fitness function model 150 (block 410). The fitness function model 150 is stored in the database 152.

The example instructions of FIG. 4 end when no additional training (e.g., retraining) is to be performed (blocks 412, 414).

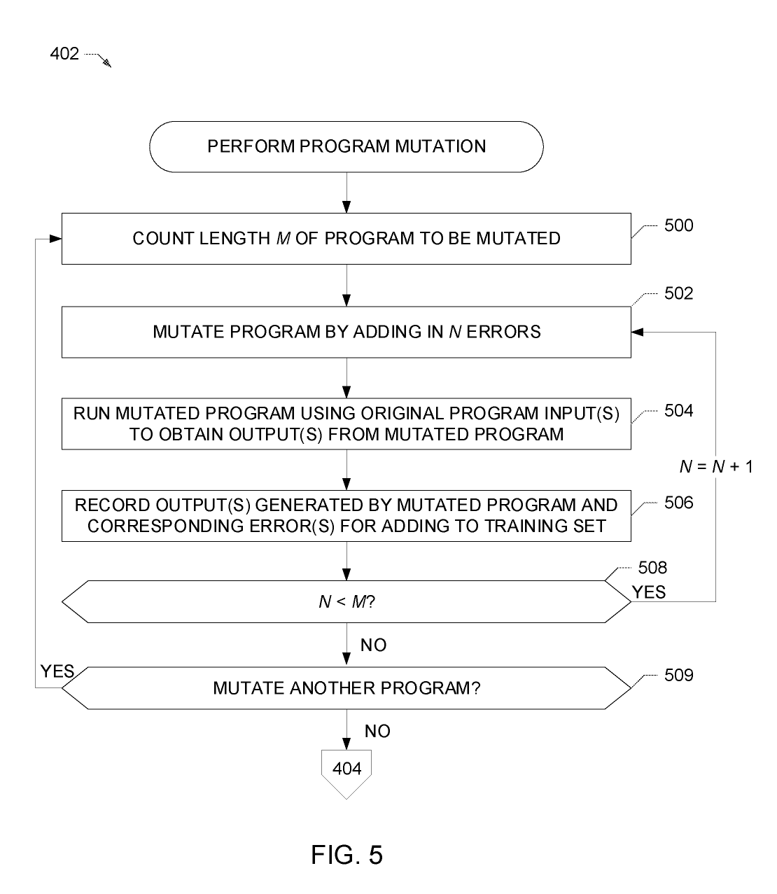

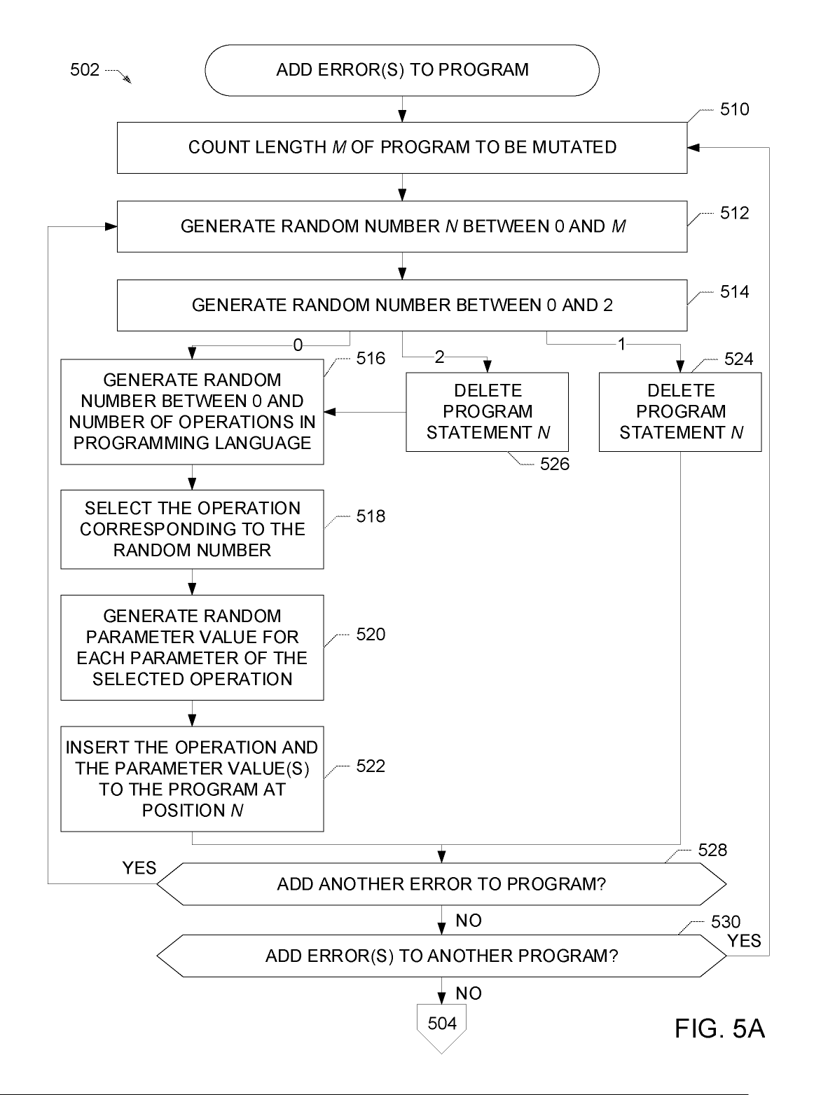

FIG. 5 is a flowchart representative of example machine readable instructions that may be executed to implement block 402 of FIG. 4 to mutate programs. The example instructions 402 can be executed by the mutator 148 of FIG. 1A.

In the example of FIG. 5, the mutator 148 counts the length m (e.g., the number of statements of executable instructions) of a program that is known to produce correct outputs for given inputs and is to be mutated (block 500). The mutator 148 mutates the program by creating copies of the program, where each program has n errors introduced into the program such that the program may no longer produce a correct output for a given input (block 502). In the example of FIG. 5, when the number of errors n equals 0, the program contains no errors and thus, can be considered to be an effective (e.g., correct) program. As another example, when the number of errors n equals 1, the program contains one error.

In the example of FIG. 5, the training controller 142 runs the mutated program containing n error(s) based on the input data that was used to obtain the correct outputs before the error(s) were introduced into the program (e.g., at blocks 400, 401 of FIG. 4) (block 504). For example, the training controller 142 runs a program containing one error (i.e., n=1) using the same input data that was used to run the program when the program had no errors (i.e., n=0). In the example of FIG. 5, the output(s) generated by the mutated program and the corresponding error number(s) are recorded (e.g., in the database 146) to be added to the training data 144 (e.g., blocks 404, 406 of FIG. 4) (block 506).

In the example of FIG. 5, the mutator determines if the number of errors n in particular program (e.g., a first program) is less than the length m (e.g., the number of statements of executable instructions) of the first program. If the number of errors n in the first program is less than the length m of the first program, the mutator 148 generates another mutated program based on the first program, where the new mutated program includes n+1 errors (block 508). Thus, in FIG. 5, the example mutator 148 creates programs including multiple errors (e.g., 2 errors, 4 errors) up the length of the program (e.g., number of statements).

When the mutator has introduced as many errors n into a particular program as the length m of the program and no further programs are to be mutated (block 509), control advances to block 404 of FIG. 4.

FIG. 5A is a flowchart representative of example machine readable instructions that may be executed to implement block 502 of FIG. 5 to add errors to a program. The example instructions 502 can be executed by the mutator 148 of FIG. 1A.

In the example of FIG. 5A, the mutator 148 counts the length m of a program to be mutated (block 510). The example mutator 148 generates a random number n between 0 and m (block 512). The mutator 148 generates a random number between 0 and 2 (block 514). In FIG. 5A, the random numbers 0, 1, and 2 are associated with an insertion error, a deletion error, and a change error, respectively.

In the example of FIG. 5A, if the random number at block 514 has a value of 0, the mutator 148 inserts an error into the program in the form of a new statement (i.e., insertion error). To insert the error, the example mutator 148 generates a random number between 0 and the number of operations in the programming language of the program (e.g., up to 40 operations) (block 516). The mutator 148 selects an operation corresponding to the randomly generated number (block 518). The mutator 148 generates a random parameter value (e.g., 0 or 1) for each parameter (e.g., one list, two list, one list/one integer) of the selected operation (block 520). In the example of FIG. 5A, the mutator 148 inserts the operation and the parameter value(s) at the position corresponding to the random number n in the program (block 522).

In the example of FIG. 5A, if the random number at block 514 has a value of 1, the mutator 148 randomly deletes a statement from the program (i.e., deletion error) (block 524). In the example of FIG. 5A, the mutator 148 deletes the statement corresponding to the random number n from the program.

In the example of FIG. 5A, if the random number at block 514 has a value of 2, the mutator 148 performs a change error in the program in which a statement is deleted (block 526) and another statement is added at the position where the deletion occurs. In the example of FIG. 5A, the mutator inserts the statements as disclosed in connection with blocks 516-522.

In the example of 5A, the assignment of the error operations to the random numbers 0, 1, or 2 are for illustrative purposes. Accordingly, different random numbers can be used and/or the random numbers 0, 1, or 2 can be associated with different error operations (e.g., the random number 0 can be associated with a deletion error and the random number 1 can be associated with an insertion error).

When no additional errors are to be added to the program (block 528) and there are no additional programs to which to add errors (block 530), control advances to block 504 of FIG. 5.

FIG. 6 is a flowchart representative of example machine readable instructions which, when executed by the example third computing system 153 of FIG. 1A, cause the example third computing system 153 to train a neural network such as the neural network implemented by the third neural network processor 154 to select a fitness function from a collection of possible fitness functions to evaluate a program. The example instructions of FIG. 6, when executed by the third computing system 153 of FIG. 1A, results in a neural network and/or a model thereof, that can be distributed to other computing systems, such as the genetic algorithm programmer 102 of FIG. 1A.

The example of FIG. 6 begins with the third training controller 158 accessing a plurality of fitness functions and known sample programs (block 600). The fitness functions can include neural network-based fitness functions (e.g., the fitness function model 150), distance-based fitness functions (e.g., Manhattan distance), etc. The programs can be known programs from prior uses or randomly generated for purposes of training. In the example of FIG. 6, the third training controller 158 identifies inputs for the programs and runs each program using the input(s) to generate corresponding output(s) (block 602, 604).

In the example of FIG. 6, the training controller 158 applies a genetic algorithm to the known sample programs to find target end result program(s) via, for instance, breeding operation(s) (block 606). The example training controller compares how the different fitness functions perform in characterizing the fitness of the known program(s) with respect to the known programs in producing a desired output for a particular input (block 608). The example training controller 158 determines how well the genetic algorithm performs in finding the target end result program(s) when a particular fitness function in the fitness function collection is used to evaluate the known programs. As a result of the analysis, the training controller 158 identifies input/output examples and an index of which fitness function appropriately (e.g., optimally) assesses a corresponding program for each input/output example.

In FIG. 6, the training controller 158 generates a training data 159 based on the input/output pairs (block 610). The training controller 158 labels the data with an indicator of the appropriate (e.g., optimal) fitness function for each input/output example (e.g., a fitness function index).

The example training controller 158 instructs the neural network trainer 156 to perform training of the neural network 154 using the training data 159 (block 612). In the example of FIG. 6, the training is based on supervised learning. As a result of the training, the neural network is trained to select a particular fitness function from a collection of possible fitness functions for a particular input/output example. The fitness function selection model 162 generated during training can be stored in the database 164 for access by, for example, the candidate program analyzer 114 of FIG. 1A (block 614).

The example instructions of FIG. 6 end when no additional training (e.g., retraining) is to be performed (blocks 616, 618).

FIG. 7 is a flowchart representative of example machine readable instructions which, when executed by the genetic algorithm programmer 102 of FIG. 1A, cause the genetic algorithm programmer 102 to generate programs based on genetic programming to identify an effective end result program (e.g., an evolved program) for target input/output data.

The example instructions of FIG. 7 begin with the random program generator 104 randomly generating candidate program(s) 106 having a length that is determined by the program length prediction model 134 developed during training of a neural network such as the neural network 122 of FIG. 1A (block 700). The program length predictor 120 executes the program length prediction model 134 for the target input/output data 108, 110 and communicates the predicted program length to the random program generator 104. In some examples, the random program generator 104 generates programs having the same length as the predicted length. In other examples, the random program generator 104 generates programs having lengths based on a program length distribution (e.g., a normal distribution, a Poisson distribution) as determined by the program length prediction model 134. The candidate program(s) 106 generated by the random program generator 104 can be stored in the database 112.

In FIG. 7, the fitness function selector 200 of the candidate program analyzer 114 selects a fitness function from a collection of possible fitness functions to evaluate the candidate program(s) 106 generated by the random program generator 104 (block 702). The candidate program analyzer 114 executes the neural network-derived fitness function selection model 162 to select a fitness function that will appropriately (e.g., optimally) assess the fitness of the candidate program(s) 106 in producing desired outputs for particular inputs. The selected fitness function can include, for example, a neural-network based fitness function, a distance metric such as Manhattan distance, etc.

In the example of FIG. 7, the fitness score calculator 202 of the candidate program analyzer 114 calculates the fitness score for the candidate program(s) 106 based on the selected fitness function. In examples in which the selected fitness function is a neural network-derived fitness function, the candidate program analyzer 114 executes the neural network-derived fitness function model 150 to determine fitness scores for the candidate program(s) generated by the random program generator 104 (block 704). Based on the fitness score(s) for the candidate program(s) 106, the candidate program analyzer 114 identifies certain ones of the candidate programs 106 that should be associated with a higher probability of being combined (e.g., crossover) and/or mutated as part of breeding operation(s) to generate effective end result program(s) (block 706). The candidate program analyzer 114 creates a biased distribution of candidate program(s) 116 that should be selected for breeding operation(s) based on the evaluation of the fitness scores.

In the example of FIG. 7, the genetic program generator 116 generates new end result program(s) 118 based on genetic programming (block 708). The genetic program generator 116 randomly selects candidate program(s) 116 for use in breeding operation(s) to generate the new end result program(s) 118. The genetic program generator 116 can generate the new end result programs via breeding operation(s) such as combining selected candidate programs, mutating selected candidate programs, etc. In the example of FIG. 7, candidate program(s) 106 associated with higher performing fitness scores as identified by the candidate program analyzer 114 have a higher probability of being selected for genetic breeding by the genetic program generator 116 over lower performing program(s). The evolved candidate programs can be stored in the database 112.

In the example of FIG. 7, the genetic program evaluator 204 of the candidate program analyzer 114 evaluates the end result program(s) 118 (e.g., the evolved candidate program(s)) generated by the genetic program generator 116 to assess whether the end result program(s) satisfy the target input/output criteria 108, 110 (block 710). The candidate program analyzer 114 determines that an end result program is effective if, for example, all input/outputs examples are satisfied by the end result program (e.g., for each given input, the end result program produces the desired output).

In examples in which the end result program(s) generated by the genetic program generator 116 are identified as being effective end result program(s) 118, the effective end result program(s) 118 can be used for training one or more neural networks (e.g., the neural networks 122, 138, 154) with respect to, for example, program length prediction and generation of a fitness function model (block 712).

In some examples, the instructions of FIG. 7 are repeated to generate additional end result programs if, for example, an effective end result program is not identified (block 714). In some examples, the additional iterations of the instructions of FIG. 7 are performed using the evolved candidate program(s) generated as a result of the breeding operations. The example instructions of FIG. 7 end when no further genetic programming is to be performed (blocks 716).

FIG. 7A is a flowchart representative of example machine readable instructions that may be executed to implement block 700 of FIG. 7. The example instructions 700 can be executed by the random program generator 104 of FIG. 1A.

When generating a new program, the number of statements n in the program has a value of 0 (block 718). In the example of FIG. 7A, the random program generator 104 determines a length m of the program to be generated (block 720). The length can be based on, for example, a predicted length based on an expected length of an effective end result program that satisfies input/output data (e.g., as determined by the program length prediction model 134 of FIG. 1A).

In the example of FIG. 7A, the random program generator 104 generates a random number between 0 and a number of operations in a programming language for the program (e.g., 40 operations) (block 722). The random program generator 104 selects the operation corresponding to the random number (block 724). For each parameter of the selected operation (e.g., one list, two lists, one list/one integer), the random program generator 104 generates a random parameter value (0 or 1) (block 726). In the example of FIG. 7A, the random program generator 104 appends the operation and the parameter value(s) to the candidate program to form the statement of the program (block 728).

When the random program generator 104 has added as many statements n as the length m of the program as determined at block 720 and no further programs are to be randomly generated (blocks 730, 732), control advances to block 702 of FIG. 7.

FIG. 8 is a flowchart representative of example machine readable instructions that, when executed by any of the first computing system 121, the second computing system 137, and/or the third computing system 153 of the example system of FIG. 1A, cause the first computing system 121, the second computing system 137, and/or the third computing system 153 to filter randomly generated programs in training data. In some examples, the training data 130, 144, 159 includes randomly generated programs. In such examples, the randomly generated programs may be evaluated to verify that, for example, the respective randomly generated programs are not producing empty lists, lists containing elements having the same values for all outputs for different inputs, or outputs having a value of 0 for all inputs. The example instructions of FIG. 8 can be executed in association with any of the example instructions of FIGS. 3-7A when generating the training data 130, 144, 159.

In the example of FIG. 8, the training controller(s) 128, 142, 158 identify input(s) for a particular randomly generated program in the training data 130, 144, 159 (block 800). The inputs can be known inputs (e.g., user-defined inputs). The example training controller(s) 128 run the program based on the inputs to generate corresponding outputs (block 802).

In the example of FIG. 8, the training controller(s) 128, 142, 158 analyze the outputs generated from running the randomly generated program based on predefined criteria. In the example of FIG. 8, the training controller(s) 128, 142, 158 determine if all of the outputs equal 0, if the outputs include an empty list, or if the outputs include a list where all the elements of the list have the same value (block 804). If the training controller(s) 128, 142, 158 determine that any of the foregoing criteria is met for the randomly generated program, the training controller(s) 128, 142, 158 remove (e.g., discard) the program from the training data 130, 144, 159 (block 806). If the training controller(s) 128, 142, 158 determine that the foregoing criteria is not met, the training controller(s) 128, 142, 158 retain the randomly generated program in the training data 130, 144, 159. Thus, the example instructions of FIG. 8 provide for filtering of automatically, randomly generated programs to remove programs that can negatively impact the training data 130, 144, 159 and/or programs that would consume resources to analyze during the neural network training but would not significantly contribute to the neural network training.

The example instructions of FIG. 8 can be repeated for each randomly generated program in the training data to determine if the program(s) should be filtered from the data (block 808). The example instructions of FIG. 8 end when there are no further programs to evaluate (block 810).

FIG. 9 is a block diagram of an example processor platform 900 structured to execute the instructions of FIGS. 3 and/or 8 to implement the first computing system of FIG. 1A. The processor platform 900 can be, for example, a server, a personal computer, a workstation, a self-learning machine (e.g., a neural network), a mobile device (e.g., a cell phone, a smart phone, a tablet such as an iPad), a personal digital assistant (PDA), an Internet appliance, or any other type of computing device.

The processor platform 900 of the illustrated example includes a processor 912. The processor 912 of the illustrated example is hardware. For example, the processor 912 can be implemented by one or more integrated circuits, logic circuits, microprocessors, GPUs, DSPs, or controllers from any desired family or manufacturer. The hardware processor may be a semiconductor based (e.g., silicon based) device. In this example, the processor implements the first neural network processor 122, the first neural network trainer 126, and the first training controller 128.

The processor 912 of the illustrated example includes a local memory 913 (e.g., a cache). The processor 912 of the illustrated example is in communication with a main memory including a volatile memory 914 and a non-volatile memory 916 via a bus 918. The volatile memory 914 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 916 may be implemented by flash memory and/or any other desired type of memory device. Access to the main memory 914, 916 is controlled by a memory controller.

The processor platform 900 of the illustrated example also includes an interface circuit 920. The interface circuit 920 may be implemented by any type of interface standard, such as an Ethernet interface, a universal serial bus (USB), a Bluetooth® interface, a near field communication (NFC) interface, and/or a PCI express interface.

In the illustrated example, one or more input devices 922 are connected to the interface circuit 920. The input device(s) 922 permit(s) a user to enter data and/or commands into the processor 912. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 924 are also connected to the interface circuit 920 of the illustrated example. The output devices 924 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 920 of the illustrated example, thus, typically includes a graphics driver card, a graphics driver chip and/or a graphics driver processor.

The interface circuit 920 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 926. The communication can be via, for example, an Ethernet connection, a digital subscriber line (DSL) connection, a telephone line connection, a coaxial cable system, a satellite system, a line-of-site wireless system, a cellular telephone system, etc.

The processor platform 900 of the illustrated example also includes one or more mass storage devices 928 for storing software and/or data. Examples of such mass storage devices 928 include floppy disk drives, hard drive disks, compact disk drives, Blu-ray disk drives, redundant array of independent disks (RAID) systems, and digital versatile disk (DVD) drives.

The machine executable instructions 932 of FIGS. 3 and/or 8 may be stored in the mass storage device 928, in the volatile memory 914, in the non-volatile memory 916, and/or on a removable non-transitory computer readable storage medium such as a CD or DVD.

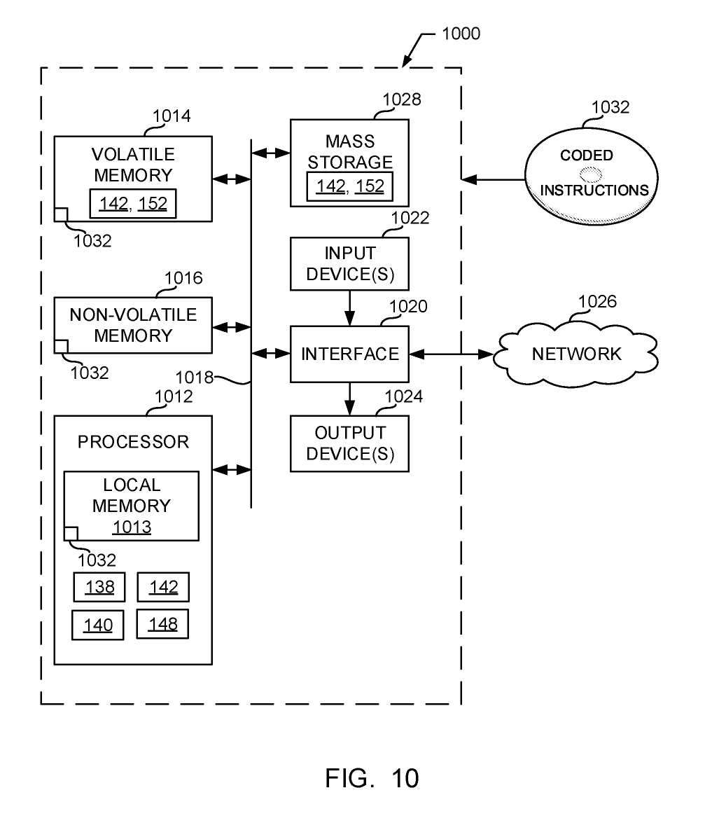

FIG. 10 is a block diagram of an example processor platform 1000 structured to execute the instructions of FIGS. 4, 5, 5A, and/or 8 to implement the second computing system of FIG. 1A. The processor platform 1000 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, or any other type of computing device.

The processor platform 1000 of the illustrated example includes a processor 1012. The processor 1012 of the illustrated example is hardware. For example, the processor 1012 can be implemented by one or more integrated circuits, logic circuits, microprocessors, GPUs, DSPs, or controllers from any desired family or manufacturer. The hardware processor may be a semiconductor based (e.g., silicon based) device. In this example, the processor implements the second neural network processor 138, the second neural network trainer 140, the second training controller 142, and the mutator 148.

The processor 1012 of the illustrated example includes a local memory 1013 (e.g., a cache). The processor 1012 of the illustrated example is in communication with a main memory including a volatile memory 1014 and a non-volatile memory 1016 via a bus 1018. The volatile memory 1014 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 1016 may be implemented by flash memory and/or any other desired type of memory device. Access to the main memory 1014, 1016 is controlled by a memory controller.

The processor platform 1000 of the illustrated example also includes an interface circuit 1020. The interface circuit 1020 may be implemented by any type of interface standard, such as an Ethernet interface, a universal serial bus (USB), a Bluetooth® interface, a near field communication (NFC) interface, and/or a PCI express interface.

In the illustrated example, one or more input devices 1022 are connected to the interface circuit 1020. The input device(s) 1022 permit(s) a user to enter data and/or commands into the processor 1012. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 1024 are also connected to the interface circuit 1020 of the illustrated example. The output devices 1024 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 1020 of the illustrated example, thus, typically includes a graphics driver card, a graphics driver chip and/or a graphics driver processor.

The interface circuit 1020 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 1026. The communication can be via, for example, an Ethernet connection, a digital subscriber line (DSL) connection, a telephone line connection, a coaxial cable system, a satellite system, a line-of-site wireless system, a cellular telephone system, etc.

The processor platform 1000 of the illustrated example also includes one or more mass storage devices 1028 for storing software and/or data. Examples of such mass storage devices 1028 include floppy disk drives, hard drive disks, compact disk drives, Blu-ray disk drives, redundant array of independent disks (RAID) systems, and digital versatile disk (DVD) drives.

The machine executable instructions 1032 of FIGS. 4, 5, 5A, and/or 8 may be stored in the mass storage device 1028, in the volatile memory 1014, in the non-volatile memory 1016, and/or on a removable non-transitory computer readable storage medium such as a CD or DVD.

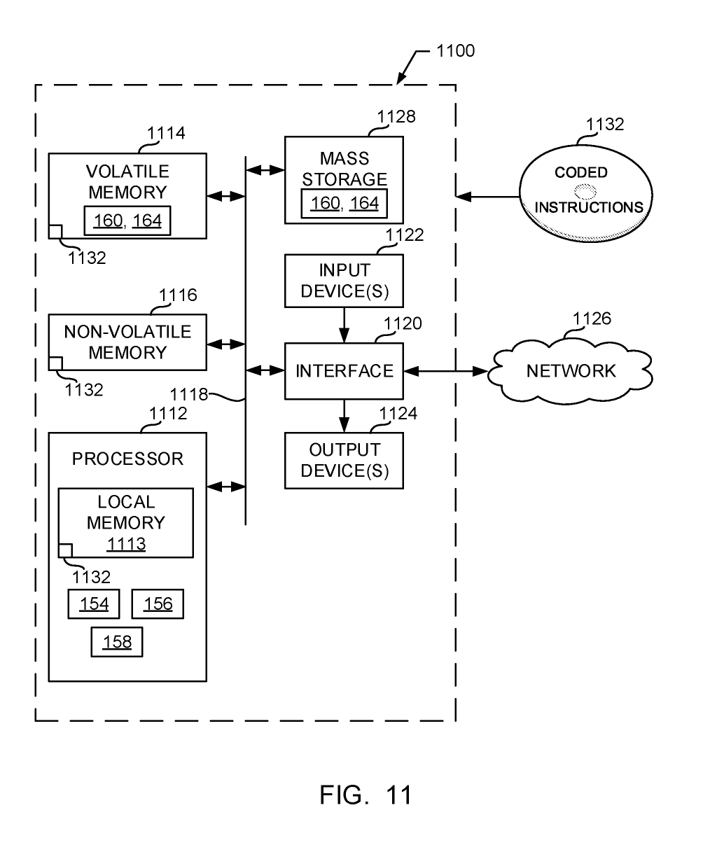

FIG. 11 is a block diagram of an example processor platform 1100 structured to execute the instructions of FIGS. 6 and/or 8 to implement the third computing system of FIG. 1A. The processor platform 1100 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, or any other type of computing device.

The processor platform 1100 of the illustrated example includes a processor 1112. The processor 1112 of the illustrated example is hardware. For example, the processor 1112 can be implemented by one or more integrated circuits, logic circuits, microprocessors, GPUs, DSPs, or controllers from any desired family or manufacturer. The hardware processor may be a semiconductor based (e.g., silicon based) device. In this example, the processor implements the third neural network processor 154, the third neural network trainer 156, and the third neural network controller 158.

The processor 1112 of the illustrated example includes a local memory 1113 (e.g., a cache). The processor 1112 of the illustrated example is in communication with a main memory including a volatile memory 1114 and a non-volatile memory 1116 via a bus 1118. The volatile memory 1114 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 1116 may be implemented by flash memory and/or any other desired type of memory device. Access to the main memory 1114, 1116 is controlled by a memory controller.

The processor platform 1100 of the illustrated example also includes an interface circuit 1120. The interface circuit 1120 may be implemented by any type of interface standard, such as an Ethernet interface, a universal serial bus (USB), a Bluetooth® interface, a near field communication (NFC) interface, and/or a PCI express interface.

In the illustrated example, one or more input devices 1122 are connected to the interface circuit 1120. The input device(s) 1122 permit(s) a user to enter data and/or commands into the processor 1112. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 1124 are also connected to the interface circuit 1120 of the illustrated example. The output devices 1124 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 1120 of the illustrated example, thus, typically includes a graphics driver card, a graphics driver chip and/or a graphics driver processor.

The interface circuit 1120 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 1126. The communication can be via, for example, an Ethernet connection, a digital subscriber line (DSL) connection, a telephone line connection, a coaxial cable system, a satellite system, a line-of-site wireless system, a cellular telephone system, etc.

The processor platform 1100 of the illustrated example also includes one or more mass storage devices 1128 for storing software and/or data. Examples of such mass storage devices 1128 include floppy disk drives, hard drive disks, compact disk drives, Blu-ray disk drives, redundant array of independent disks (RAID) systems, and digital versatile disk (DVD) drives.

The machine executable instructions 1132 of FIGS. 6 and/or 8 may be stored in the mass storage device 1128, in the volatile memory 1114, in the non-volatile memory 1116, and/or on a removable non-transitory computer readable storage medium such as a CD or DVD.

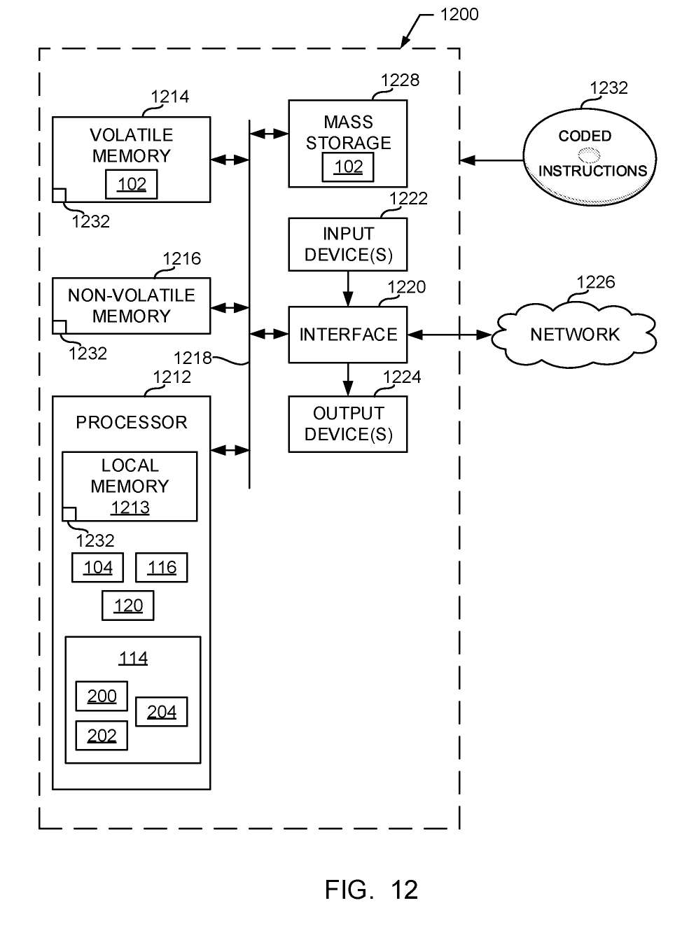

FIG. 12 is a block diagram of an example processor platform 1200 structured to execute the instructions of FIGS. 7 and/or 7A to implement the genetic algorithm programmer of FIGS. 1 and 2. The processor platform 1200 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, or any other type of computing device.

The processor platform 1200 of the illustrated example includes a processor 1212. The processor 1212 of the illustrated example is hardware. For example, the processor 1212 can be implemented by one or more integrated circuits, logic circuits, microprocessors, GPUs, DSPs, or controllers from any desired family or manufacturer. The hardware processor may be a semiconductor based (e.g., silicon based) device. In this example, the processor implements the random program generator 104, the candidate program analyzer 114, the genetic program generator 116, the program length predictor 120, the fitness function selector 200, the fitness score calculator 202, and the genetic program evaluator 204.

The processor 1212 of the illustrated example includes a local memory 1213 (e.g., a cache). The processor 1212 of the illustrated example is in communication with a main memory including a volatile memory 1214 and a non-volatile memory 1216 via a bus 1218. The volatile memory 1214 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 1216 may be implemented by flash memory and/or any other desired type of memory device. Access to the main memory 1214, 1216 is controlled by a memory controller.

The processor platform 1200 of the illustrated example also includes an interface circuit 1220. The interface circuit 1220 may be implemented by any type of interface standard, such as an Ethernet interface, a universal serial bus (USB), a Bluetooth® interface, a near field communication (NFC) interface, and/or a PCI express interface.

In the illustrated example, one or more input devices 1222 are connected to the interface circuit 1220. The input device(s) 1222 permit(s) a user to enter data and/or commands into the processor 1212. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 1224 are also connected to the interface circuit 1220 of the illustrated example. The output devices 1224 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 1220 of the illustrated example, thus, typically includes a graphics driver card, a graphics driver chip and/or a graphics driver processor.

The interface circuit 1220 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 1226. The communication can be via, for example, an Ethernet connection, a digital subscriber line (DSL) connection, a telephone line connection, a coaxial cable system, a satellite system, a line-of-site wireless system, a cellular telephone system, etc.

The processor platform 1200 of the illustrated example also includes one or more mass storage devices 1228 for storing software and/or data. Examples of such mass storage devices 1228 include floppy disk drives, hard drive disks, compact disk drives, Blu-ray disk drives, redundant array of independent disks (RAID) systems, and digital versatile disk (DVD) drives.

The machine executable instructions 1232 of FIGS. 7 and/or 7A may be stored in the mass storage device 1228, in the volatile memory 1214, in the non-volatile memory 1216, and/or on a removable non-transitory computer readable storage medium such as a CD or DVD.

From the foregoing, it will be appreciated that example apparatus, systems, and methods have been disclosed that use machine learning to more efficiently identify effective end result or evolved programs created via genetic algorithm-based program synthesis. In some examples disclosed herein, a neural network is trained to predict a length of an effective end result program that satisfies given input/output criteria. The predicted length is used to generate candidate programs having the predicted length and/or a length similar to the predicted length. As a result, the efficiency of the genetic programming process in generating effective end result program(s) is increased because the candidate programs used to generate end result program(s) are more closely aligned with the predicted program length, and programs outside the length criteria are not considered (e.g., are discarded), thereby saving processing resources. In some other examples disclosed herein, a neural network is trained to select an appropriate (e.g., optimal) fitness function from a collection of fitness functions for evaluating a candidate program. Thus, rather than applying a single fitness function to evaluate all candidate programs, an appropriate fitness function that fits the criteria can be selected based on the program and the target input/output data. In some examples disclosed herein, a neural network is trained to produce a fitness function that can identify deeper (e.g., non-linear) similarities between inputs and outputs that may be too subtle for a user to identify and that provides for more accurate evaluation of a candidate program than some known fitness functions based on distance metrics.

Disclosed methods, apparatus and articles of manufacture improve the efficiency of using a computing device by using machine learning to more efficiently generate and evaluate new programs during program synthesis. Examples disclosed herein remove or reduce inputs provided by a human user by employing neural networks to select an appropriate fitness function; to generate a more robust fitness function to evaluate program fitness or correctness; and/or to predict a program length rather than relying on prior knowledge provided by a user with respect to program length, which may not be inaccurate or feasible in view of the use of program synthesis to generate new programs. Thus, disclosed methods, apparatus and articles of manufacture are directed to one or more improvement(s) in the functioning of a computer.

The following paragraphs provide various examples disclosed herein.

Example 1 includes an apparatus including a program length predictor to predict a length of a first program by executing a first neural network model and a program generator to generate a first candidate program having a length corresponding to the predicted length. The example apparatus includes a candidate program analyzer to generate a fitness score for the first candidate program by executing a second neural network model and identify the first candidate program for use in a breeding operation relative a second candidate program based on the fitness score. The example apparatus includes a genetic program generator to perform the breeding operation with at least one of the first candidate program or the second candidate program to generate an evolved candidate program.

Example 2 includes the apparatus of example 1, wherein the second neural network model implements a fitness function.

Example 3 includes the apparatus of example 1, wherein the candidate program analyzer is to select a fitness function to generate the fitness score by executing a third neural network.

Example 4 includes the apparatus of example 1, wherein the breeding operation includes one or more of mutation of one or more respective parameters of the at least one of the first candidate program or the second candidate program or crossover of the one or more parameters of the first candidate program with the one or more parameters of the second candidate program or with one or more parameters of another program.

Example 5 includes the apparatus of example 1, wherein the second candidate program has a length corresponding to the predicted length.

Example 6 includes the apparatus of example 5, wherein the genetic program generator is to randomly select the at least one of the first candidate program or the second candidate program with which to perform the breeding operation.

Example 7 includes the apparatus of example 1, wherein the candidate program analyzer is to evaluate the evolved candidate program based on input data and output data.

Example 8 includes the apparatus of example 7, wherein the first neural network model is to be retrained based on the evolved candidate program.

Example 9 includes the apparatus of example 7, wherein the program generator is to generate another candidate program in response to the evaluation of the evolved candidate program.

Example 10 includes at least one non-transitory computer-readable medium comprising instructions that, when executed, cause at least one processor to at least generate a first neural network model to predict a length of an end result program, the end result program to be selected from at least one of a first candidate program or a second candidate program, the at least one of the first candidate program or the second candidate program to be generated based on a genetic algorithm; and generate a second neural network model implementing a fitness function to evaluate the first candidate program and the second candidate program, at least one of the first neural network model or the second neural network model to be used to generate the end result program.

Example 11 includes the at least one non-transitory computer-readable medium of example 10, wherein the instructions, when executed, cause the at least one processor to generate the first neural network model based on training data including inputs, corresponding outputs, and respective lengths of programs that produce the outputs based on the inputs.

Example 12 includes the at least one non-transitory computer-readable medium of example 10, wherein the first neural network model is to predict a program length distribution.

Example 13 includes the at least one non-transitory computer-readable medium of example 10, wherein the instructions, when executed, cause the at least one processor to run a first program using an input to obtain a corresponding first output; create a mutation of the first program to generate a mutated first program; run the mutated program using the input to obtain a second output; and generate the second neural network model based on the input, the first output, and the second output.

Example 14 includes the at least one non-transitory computer-readable medium of example 13, wherein the instructions, when executed, cause the at least one processor to create the mutation by at least one of removing a statement from the first program or adding a statement to the first program.

Example 15 includes the at least one non-transitory computer-readable medium of example 10, wherein the fitness function is a first fitness function and the instructions, when executed, cause the at least one processor to generate a third neural network model to select the first fitness function or a second fitness function to evaluate the first candidate program and the second candidate program.

Example 16 includes the at least one non-transitory computer-readable medium of example 15, wherein the second fitness function is a distance-based fitness function.

Example 17 includes The at least one non-transitory computer-readable medium of example 10, wherein the instructions, when executed, cause the at least one processor to generate the first neural network using training data, the training data including a plurality of programs.

Example 18 includes the at least one non-transitory computer-readable medium of example 17, wherein the instructions, when executed, cause the at least one processor to run the plurality of programs based on inputs to generate outputs; and filter respective ones of the programs based on the outputs.

Example 19 includes an apparatus comprising means for predicting a length of a first program; means for generating a first candidate program having a length corresponding to the predicted length; means for generating a fitness score for the candidate program by executing a neural network model, the means for generating the fitness score to identify the first candidate program for use in a breeding operation relative a second candidate program based on the fitness score; and means for generating an evolved candidate program with at least one of the first candidate program or the second candidate program by performing a breeding operation.

Example 20 includes the apparatus of example 19, wherein neural network model is a first neural network model and the means for predicting is to predict the length by executing a second neural network model.

Example 21 includes the apparatus of example 19, wherein the neural network model implements a fitness function.

Example 22 includes the apparatus of example 21, where the means for generating a fitness score is to select the fitness function by executing a third neural network model.

Example 23 includes the apparatus of example 19, wherein the means for generating the evolved candidate program is to perform the breeding operation by mutating one or more respective parameters of the at least one of the first candidate program or the second candidate program.

Example 24 includes the apparatus of example 19, further including means for evaluating the evolved candidate program.

Example 25 includes the apparatus of example 24, wherein the means for generating is to generate another candidate program in response to the evaluation of the evolved candidate program.

Example 26 includes a method including predicting, by executing an instruction with a processor, a length of a first program by executing a first neural network model; generating, by executing an instruction with the processor, a candidate program having a length corresponding to the predicted length; calculating, by executing an instruction with the processor, a fitness score for the candidate program by executing a second neural network model; identifying, by executing an instruction with the processor, the first candidate program for use in a breeding operation relative a second candidate program based on the fitness score; and performing, by executing an instruction with the processor, a breeding operation with at least one of the first candidate program or the second candidate program to generate an evolved candidate program.

Example 27 includes the method of example 26, wherein the second neural network model implements a fitness function.

Example 28 includes the method of example 26, further including selecting a fitness function to generate the fitness score by executing a third neural network.

Example 29 includes the method of example 26, wherein the breeding operation includes one or more of mutation of one or more respective parameters of the at least one for the first candidate program or the second candidate program or crossover of the one or more parameters of the first candidate program with the one or more parameters of the second candidate program or one or more parameters of another candidate program.

Example 30 includes the method of example 26, wherein the second candidate program has a length corresponding to the predicted length.

Example 31 includes the method of example 30, further including randomly selecting the at least one of the first candidate program or the second candidate program with which to perform the breeding operation.

Example 32 includes the method of example 26, further including evaluating the evolved candidate program based on input data and output data.

Example 33 includes the method of example 32, further including generating another candidate program in response to the evaluation of the evolved candidate program.

Example 34 includes the method of example 32, further including retraining the first neural network model based on the evolved candidate program.

Example 35 includes at least one non-transitory computer-readable medium comprising instructions that, when executed, cause at least one processor to at least predict a length of a first program by executing a first neural network model; generate a candidate program having a length corresponding to the predicted length; generate a fitness score for the candidate program by executing a second neural network model; identify the first candidate program for use in a breeding operation relative a second candidate program based on the fitness score; and perform the breeding operation with at least one of the first candidate program or the second candidate program to generate an evolved candidate program.

Example 36 includes the at least one non-transitory computer-readable medium of example 35, wherein the second neural network model implements a fitness function.

Example 37 includes the at least one non-transitory computer-readable medium of example 35, wherein the instructions, when executed, cause the at least one processor to select a fitness function to generate the fitness score by executing a third neural network.

Example 38 includes the at least one non-transitory computer-readable medium of example 35, wherein the breeding operation includes one or more of mutation of one or more respective parameters of the at least one of the first candidate program or the second candidate program or crossover of the one or more parameters of the first candidate program with the one or more parameters of the second candidate program or with one or more parameters of another program.

Example 39 includes the at least one non-transitory computer-readable medium of example 35, wherein the second candidate program has a length corresponding to the predicted length.

Example 40 includes the at least one non-transitory computer-readable medium of example 39, wherein the instructions, when executed, cause the at least one processor to randomly select the at least one of the first candidate program or the second candidate program with which to perform the breeding operation.

Example 41 includes the at least one non-transitory computer-readable medium of example 35, wherein the instructions, when executed, cause the at least one processor to evaluate the evolved candidate program based on input data and output data.

Example 42 includes the at least one non-transitory computer-readable medium of example 35, wherein the instructions, when executed, cause the at least one processor to retrain the first neural network based on the evolved candidate program.

Example 43 includes the at least one non-transitory computer-readable medium of example 35, wherein the instructions, when executed, cause the at least one processor to generate another candidate program in response to the evaluation of the evolved candidate program.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

1. An apparatus comprising:
a program length predictor to predict a length of a first program by executing a first neural network model;
a program generator to generate a first candidate program having a length corresponding to the predicted length;
a candidate program analyzer to:
generate a fitness score for the first candidate program by executing a second neural network model; and
identify the first candidate program for use in a breeding operation relative a second candidate program based on the fitness score; and
a genetic program generator to perform the breeding operation with at least one of the first candidate program or the second candidate program to generate an evolved candidate program.

2. The apparatus of claim 1, wherein the second neural network model implements a fitness function.

3. The apparatus of claim 1, wherein the candidate program analyzer is to select a fitness function to generate the fitness score by executing a third neural network.

4. The apparatus of claim 1, wherein the breeding operation includes one or more of mutation of one or more respective parameters of the at least one of the first candidate program or the second candidate program or crossover of the one or more parameters of the first candidate program with the one or more parameters of the second candidate program or with one or more parameters of another program.

5. The apparatus of claim 1, wherein the second candidate program has a length corresponding to the predicted length.

6. The apparatus of claim 5, wherein the genetic program generator is to randomly select the at least one of the first candidate program or the second candidate program with which to perform the breeding operation.

7. The apparatus of claim 1, wherein the candidate program analyzer is to evaluate the evolved candidate program based on input data and output data.

8. (canceled)

9. (canceled)

10. At least one non-transitory computer-readable medium comprising instructions that, when executed, cause at least one processor to at least:
generate a first neural network model to predict a length of an end result program, the end result program to be selected from at least one of a first candidate program or a second candidate program, the at least one of the first candidate program or the second candidate program to be generated based on a genetic algorithm; and
generate a second neural network model implementing a fitness function to evaluate the first candidate program and the second candidate program, at least one of the first neural network model or the second neural network model to be used to generate the end result program.

11. The at least one non-transitory computer-readable medium of claim 10, wherein the instructions, when executed, cause the at least one processor to generate the first neural network model based on training data including inputs, corresponding outputs, and respective lengths of programs that produce the outputs based on the inputs.

12. The at least one non-transitory computer-readable medium of claim 10, wherein the first neural network model is to predict a program length distribution.

13. The at least one non-transitory computer-readable medium of claim 10, wherein the instructions, when executed, cause the at least one processor to:
run a first program using an input to obtain a corresponding first output;
create a mutation of the first program to generate a mutated first program;
run the mutated program using the input to obtain a second output; and
generate the second neural network model based on the input, the first output, and the second output.

14. The at least one non-transitory computer-readable medium of claim 13, wherein the instructions, when executed, cause the at least one processor to create the mutation by at least one of removing a statement from the first program or adding a statement to the first program.

15. The at least one non-transitory computer-readable medium of claim 10, wherein the fitness function is a first fitness function and the instructions, when executed, cause the at least one processor to generate a third neural network model to select the first fitness function or a second fitness function to evaluate the first candidate program and the second candidate program.

16. (canceled)

17. (canceled)

18. (canceled)

19. An apparatus comprising:
means for predicting a length of a first program;
means for generating a first candidate program having a length corresponding to the predicted length;
means for generating a fitness score for the candidate program by executing a neural network model, the means for generating the fitness score to identify the first candidate program for use in a breeding operation relative a second candidate program based on the fitness score; and
means for generating an evolved candidate program with at least one of the first candidate program or the second candidate program by performing a breeding operation.

20. The apparatus of claim 19, wherein neural network model is a first neural network model and the means for predicting is to predict the length by executing a second neural network model.

21. The apparatus of claim 19, wherein the neural network model implements a fitness function.

22. The apparatus of claim 21, where the means for generating a fitness score is to select the fitness function by executing a third neural network model.

23. The apparatus of claim 19, wherein the means for generating the evolved candidate program is to perform the breeding operation by mutating one or more respective parameters of the at least one of the first candidate program or the second candidate program.

24. The apparatus of claim 19, further including means for evaluating the evolved candidate program.

25. The apparatus of claim 24, wherein the means for generating is to generate another candidate program in response to the evaluation of the evolved candidate program.

26.-43. (canceled)
